(19) United States
(12) Patent Application Publication
(10) Pub. No.: US 2011/0053925 A1
(43) Pub. Date: Mar. 3, 2011

(54) Hydroxamate-Based Inhibitors of Deacetylases

(75) Inventors: Young Shin CHO, Cambridge, MA (US); Lei JIANG, Shanghai (CN); Michael SHULTZ, Stow, MA (US); Christine Hiu-Tung CHEN, Waltham, MA (US); Gang LIU, Waltham, MA (US); Jianke LI, Hamden, CT (US)

(73) Assignee: Novartis AG

(21) Appl. No.: 12/870,123

(22) Filed: Aug. 27, 2010

Related U.S. Application Data
(60) Provisional application No. 61/237,755, filed on Aug. 28, 2009.

(51) Int. Cl. A61K 31/537; C12N 5/00; A61K 31/5377; A61K 31/495; A61K 31/438; A61K 31/437; A61K 31/454; C07D 498/10; C07D 413/14; C07D 413/02; C07D 295/205; C07D 471/10; C07D 471/04; C07D 401/02; A61P 25/16; A61P 25/28; A61P 9/04; A61P 7/02; A61P 19/02; A61P 9/10; A61P 35/00

(52) U.S. Cl. 514/230.5; 435/375; 514/235.2; 514/236.8; 514/255.01; 514/278; 514/292; 514/323; 544/71; 544/130; 544/137; 544/138; 544/389; 546/20; 546/87; 546/201

(57) ABSTRACT

The present teachings relate to compounds of Formula I:

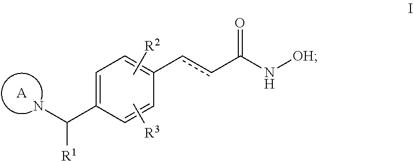

and pharmaceutically acceptable salts, hydrates, esters, and prodrugs thereof, wherein R¹, R², R³, ring A, and [symbol] are as defined herein.

The present teachings also provide methods of preparing compounds of Formula I and methods of using compounds of Formula I in treating, inhibiting, or preventing pathologic conditions or disorders mediated wholly or in part by deacetylases.

### BACKGROUND OF THE INVENTION

Deacetylation, catalyzed by deacetylases, relates to transcriptional regulation of proteins involved in signal transduction. Accordingly, deacetylase inhibitors can be used for the therapy of pathological conditions or disorders wholly or in part mediated by one or more deacetylases. These conditions or disorders can include retinopathies, age-related macula degeneration, psoriasis, haemangioblastoma, haemangioma, arteriosclerosis, muscle wasting conditions such as muscular dystrophies, cachexia, Huntington's syndrome, inflammatory diseases such as rheumatoid or rheumatic inflammatory diseases, and neoplastic diseases. More specifically, deacetylase inhibitors can be useful for treating arthritis and arthritic conditions (e.g., osteoarthritis, rheumatoid arthritis, and the like), other chronic inflammatory disorders (e.g., chronic asthma, arterial or post-transplantational atherosclerosis, endometriosis, and the like), solid tumors (e.g., cancers of the gastrointestinal tract, pancreas, breast, stomach, cervix, bladder, kidney, prostate, esophagus, ovaries, endometrium, lung, brain, melanoma, Kaposi's sarcoma, squamous cell carcinoma of head and neck, malignant pleural mesotherioma, lymphoma, multiple myeloma, and the like), and liquid tumors (e.g., leukemias).

More specifically, histone deacetylases remove an acetyl group from an N-acetyl lysine on a histone. In normal cells, histone deacetylase (HDAC) and histone acetyltransferase together control the level of acetylation of histones to maintain a balance. Reversible acetylation of histones is a major regulator of gene expression that acts by altering accessibility of transcription factors to DNA.

HDAC inhibitors have been studied for their therapeutic effect to proliferative diseases, including tumors, hyperproliferative conditions, neoplasias, immune diseases, and central and peripheral nervous system diseases. More specifically, HDAC inhibitors can be useful for their antitumor activities. For example, butyric acid and its derivatives, including sodium phenylbutyrate, have been reported to induce apoptosis in vitro in human colon carcinoma, leukemia, and retinoblastoma cell lines. However, butyric acid and its derivatives are not useful as pharmacological agents because they tend to be metabolized rapidly and have a very short half-life in vivo. Other HDAC inhibitors that have been studied for their anti-cancer activities include trichostatin A and trapoxin. Trichostatin A, an antifungal and antibiotic agent, is a reversible inhibitor of mammalian HDAC and trapoxin, a cyclic tetrapeptide, is an irreversible inhibitor of mammalian HDAC. Although trichostatin and trapoxin have been studied for their anti-cancer activities, the in vivo instability of these compounds makes them less suitable as anti-cancer drugs.

### SUMMARY

The present teachings relate to compounds of Formula I:

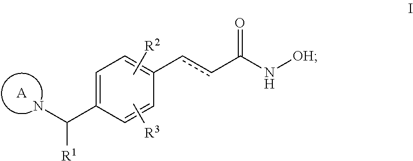

and pharmaceutically acceptable salts, hydrates, esters, and prodrugs thereof, where R¹, R², R³, ring A, and [symbol] are as defined herein.

The present teachings also relate to methods of preparing compounds of Formula I, including pharmaceutically acceptable salts, hydrates, esters and prodrugs thereof, and methods of using compounds of Formula I, including pharmaceutically acceptable salts, hydrates, esters and prodrugs thereof, in treating pathologic conditions or disorders mediated wholly or in part by deacetylases, for example, including administering a therapeutically effective amount of a compound of Formula I to a patient, for example, a patient in need thereof. Examples of the pathologic conditions or disorders include undesired proliferative conditions, neurodegenerative diseases, cardiovascular diseases, strokes, autoimmune diseases, inflammatory diseases, undesired immunological processes, and fungal infections.

The foregoing as well as other features and advantages of the present teachings will be more fully understood from the following description and claims.

### DETAILED DESCRIPTION

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components.

The use of the term “include,” “includes,” “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±5% variation from the nominal value.

It should be understood that the order of steps or order for performing certain actions is immaterial so long as the present teachings remain operable. Moreover, two or more steps or actions may be conducted simultaneously.

As used herein, a “compound” refers to the compound itself and its pharmaceutically acceptable salts, hydrates, and esters, unless otherwise understood from the context of the description or expressly limited to one particular form of the compound, i.e., the compound itself, or a pharmaceutically acceptable salt, hydrate, or ester thereof.

As used herein, “halo” or “halogen” refers to fluoro, chloro, bromo, and iodo.

As used herein, “oxo” refers to a double-bonded oxygen (i.e., ═O).

As used herein, “alkyl” refers to a straight-chain or branched saturated hydrocarbon group. In some embodiments, an alkyl group can have from 1 to 10 carbon atoms (e.g., from 1 to 6 carbon atoms). Examples of alkyl groups include methyl (Me), ethyl (Et), propyl (e.g., n-propyl and isopropyl), butyl (e.g., n-butyl, isobutyl, s-butyl, t-butyl), pentyl groups (e.g., n-pentyl, isopentyl, neopentyl), and the like. In some embodiments, alkyl groups optionally can be substituted with up to four groups independently selected from -L-R⁸ and -L-R¹³, where L, R⁸, and R¹³ are as described herein. A lower alkyl group typically has up to 4 carbon atoms. Examples of lower alkyl groups include methyl, ethyl, propyl (e.g., n-propyl and isopropyl), and butyl groups (e.g., n-butyl, isobutyl, s-butyl, t-butyl).

As used herein, “alkenyl” refers to a straight-chain or branched alkyl group having one or more carbon-carbon double bonds. In some embodiments, an alkenyl group can have from 2 to 10 carbon atoms (e.g., from 2 to 6 carbon atoms). Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In some embodiments, alkenyl groups optionally can be substituted with up to four groups independently selected from -L-R⁸ and -L-R¹³, where L, R⁸, and R¹³ are as described herein.

As used herein, “alkynyl” refers to a straight-chain or branched alkyl group having one or more carbon-carbon triple bonds. In some embodiments, an alkynyl group can have from 2 to 10 carbon atoms (e.g., from 2 to 6 carbon atoms). Examples of alkynyl groups include ethynyl, propynyl, butyryl, pentynyl, and the like. The one or more carbon-carbon triple bonds can be internal (such as in 2-butyne) or terminal (such as in 1-butyne). In some embodiments, alkynyl groups optionally can be substituted with up to four groups independently selected from -L-R⁸ and -L-R¹³, where L, R⁸, and R¹³ are as described herein.

As used herein, “alkoxy” refers to an —O-alkyl group. Examples of alkoxy groups include methoxy, ethoxy, propoxy (e.g., n-propoxy and isopropoxy), t-butoxy groups, and the like.

As used herein, “alkylthio” refers to an —S— alkyl group. Examples of alkylthio groups include methylthio, ethylthio, propylthio (e.g., n-propylthio and isopropylthio), t-butylthio groups, and the like.

As used herein, “haloalkyl” refers to an alkyl group having one or more halogen substituents. In some embodiments, a haloalkyl group can have 1 to 10 carbon atoms (e.g., from 1 to 6 carbon atoms). Examples of haloalkyl groups include CF₃, C₂F₅, CHF₂, CH₂F, CCl₃, CHCl₂, CH₂Cl, C₂Cl₅, and the like. Perhaloalkyl groups, i.e., alkyl groups where all of the hydrogen atoms are replaced with halogen atoms (e.g., CF₃ and C₂F₅), are included within the definition of “haloalkyl.” For example, a C₁₋₁₀ haloalkyl group can have the formula —CᵢH₂ᵢ₊₁₋ⱼXⱼ, where X is F, Cl, Br, or I, i is an integer in the range of 1 to 10, and j is an integer in the range of 0 to 21, provided that j is less than or equal to 2i+1.

As used herein, “cycloalkyl” refers to a non-aromatic carbocyclic group including cyclized alkyl, alkenyl, and alkynyl groups. A cycloalkyl group can be monocyclic (e.g., cyclohexyl) or polycyclic (e.g., containing fused, bridged, and/or spiro ring systems), where the carbon atoms are located inside or outside of the ring system. A cycloalkyl group, as a whole, can have from 3 to 14 ring atoms (e.g., from 3 to 8 carbon atoms for a monocyclic cycloalkyl group and from 7 to 14 carbon atoms for a polycyclic cycloalkyl group). Any suitable ring position of the cycloalkyl group can be covalently linked to the defined chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like. In some embodiments, cycloalkyl groups optionally can be substituted with up to four groups independently selected from -L-R⁸ and -L-R¹³, where L, R⁸, and R¹³ are as described herein. For example, cycloalkyl groups can be substituted with one or more oxo groups.

As used herein, “heteroatom” refers to an atom of any element other than carbon or hydrogen and includes, for example, nitrogen, oxygen, sulfur, phosphorus, and selenium.

As used herein, “cycloheteroalkyl” refers to a non-aromatic cycloalkyl group that contains at least one (e.g., one, two, three, four, or five) ring heteroatom selected from O, N, and S, and optionally contains one or more (e.g., one, two, or three) double or triple bonds. A cycloheteroalkyl group, as a whole, can have from 3 to 14 ring atoms and contains from 1 to 5 ring heteroatoms (e.g., from 3-6 ring atoms for a monocyclic cycloheteroalkyl group and from 7 to 14 ring atoms for a polycyclic cycloheteroalkyl group). The cycloheteroalkyl group can be covalently attached to the defined chemical structure at any heteroatom(s) or carbon atom(s) that results in a stable structure. One or more N or S atoms in a cycloheteroalkyl ring may be oxidized (e.g., morpholine N-oxide, thiomorpholine S-oxide, thiomorpholine S,S-dioxide). In some embodiments, nitrogen atoms of cycloheteroalkyl groups can bear a substituent, for example, a -L-R⁸ or -L-R¹³ group, where L, R⁸, and R¹³ are as described herein. Cycloheteroalkyl groups can also contain one or more oxo groups, such as phthalimidyl, piperidonyl, oxazolidinonyl, 2,4(1H,3H)-dioxo-pyrimidinyl, pyridin-2(1H)-onyl, and the like. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothienyl, piperidinyl, piperazinyl, and the like. In some embodiments, cycloheteroalkyl groups optionally can be substituted with up to four groups independently selected from -L-R⁸ and -L-R¹³, where L, R⁸, and R¹³ are as described herein.

As used herein, “aryl” refers to an aromatic monocyclic hydrocarbon ring system or a polycyclic ring system where at least one of the rings in the ring system is an aromatic hydrocarbon ring and any other aromatic rings in the ring system include only hydrocarbons. In some embodiments, a monocyclic aryl group can have from 6 to 14 carbon atoms and a polycyclic aryl group can have from 8 to 14 carbon atoms. The aryl group can be covalently attached to the defined chemical structure at any carbon atom(s) that result in a stable structure. In some embodiments, an aryl group can have only aromatic carbocyclic rings, e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cycloalkyl or cycloheteroalkyl rings. Examples of such aryl groups include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like. In some embodiments, each aryl group optionally can be substituted with up to four groups independently selected from -L-R⁸ and -L-R¹³, where L, R⁸, and R¹³ are as described herein.

As used herein, “heteroaryl” refers to an aromatic monocyclic ring system containing at least one ring heteroatom selected from O, N, and S or a polycyclic ring system where at least one of the rings in the ring system is aromatic and contains at least one ring heteroatom. A heteroaryl group, as a whole, can have from 5 to 14 ring atoms and contain 1-5 ring heteroatoms. In some embodiments, heteroaryl groups can include monocyclic heteroaryl rings fused to one or more aromatic carbocyclic rings, non-aromatic carbocyclic rings, or non-aromatic cycloheteroalkyl rings. The heteroaryl group can be covalently attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5-membered and 6-membered monocyclic and 5-6 bicyclic ring systems shown below:

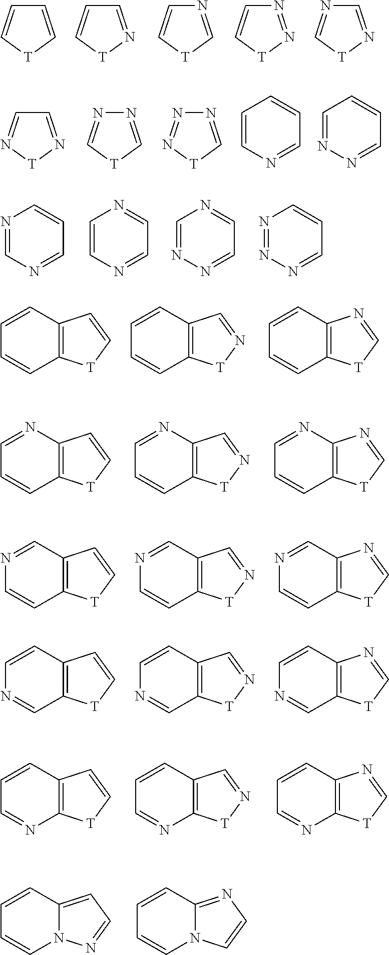

where T is O, S, NH, N-L-R⁸, or N-L-R¹³, where L, R⁸, and R¹³ are as defined herein. Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In some embodiments, heteroaryl groups can be substituted with up to four groups independently selected from -L-R⁸ and -L-R¹³, where L, R⁸, and R¹³ are as described herein.

The compounds of the present teachings can include a “divalent group” defined herein as a linking group capable of forming a covalent bond with two other moieties. For example, compounds described herein can include a divalent C₁₋₁₀ alkyl group, such as, for example, a methylene group.

As used herein, a “leaving group” (“LG”) refers to a charged or uncharged atom (or group of atoms) that can be displaced as a stable species as a result of, for example, a substitution or elimination reaction. Examples of leaving groups include, but are not limited to, halide (e.g., Cl, Br, I), azide (N₃), thiocyanate (SCN), nitro (NO₂), cyanate (CN), tosylate (toluenesulfonate, OTs), mesylate (methanesulfonate, OMs), brosylate (p-bromobenzenesulfonate, OBs), nosylate (4-nitrobenzenesulfonate, ONs), water (H₂O), ammonia (NH₃), and triflate (trifluoromethanesulfonate, OTf).

At various places in the present specification, substituents of compounds are disclosed in groups or in ranges. It is specifically intended that the description includes each and every individual subcombination of the members of such groups and ranges. For example, the term “C₁₋₁₀ alkyl” is specifically intended to individually disclose C₁, C₂, C₃, C₄, C₅, C₆, C₇, C₈, C₉, C₁₀, C₁-C₁₀, C₁-C₉, C₁-C₈, C₁-C₇, C₁-C₆, C₁-C₅, C₁-C₄, C₁-C₃, C₁-C₂, C₂-C₁₀, C₂-C₉, C₂-C₈, C₂-C₇, C₂-C₆, C₂-C₅, C₂-C₄, C₂-C₃, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉, and C₉-C₁₀ alkyl. By way of another example, the term “5-14 membered heteroaryl group” is specifically intended to individually disclose a heteroaryl group having 5, 6, 7, 8, 9, 10, 11, 12, 13, 14, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, or 13-14 ring atoms; and the phrase “optionally substituted with 1-4 groups” is specifically intended to individually disclose a chemical group that can include 0, 1, 2, 3, 4, 0-4, 0-3, 0-2, 0-1, 1-4, 1-3, 1-2, 2-4, 2-3, and 3-4 groups.

Compounds described herein can contain an asymmetric atom (also referred as a chiral center) and some of the compounds can contain two or more asymmetric atoms or centers, which can thus give rise to optical isomers (enantiomers) and diastereomers (geometric isomers). Compounds of the present teachings include such optical isomers and diastereomers in their respective enantiomerically pure forms (i.e., (+) and (−) stereoisomers), in racemic mixtures, and in other mixtures of the (+) and (−) stereoisomers, as well as pharmaceutically acceptable salts, hydrates, and esters thereof. Optical isomers in pure form or in enantiomerically enriched mixture can be obtained by standard procedures known to those skilled in the art, which include, but are not limited to, chiral separation, diastereomeric salt formation, kinetic resolution, and asymmetric synthesis. The present teachings also encompass cis and trans-isomers of compounds containing alkenyl moieties (e.g., alkenes and imines). It is also understood that the present teachings encompass all possible regioisomers and mixtures thereof, which can be obtained in pure form or in substantially enriched mixture by standard separation procedures known to those skilled in the art, including, but are not limited to, column chromatography, thin-layer chromatography, simulated moving-bed chromatography, and high-performance liquid chromatography.

In one aspect, the present teachings provide compounds of Formula I:

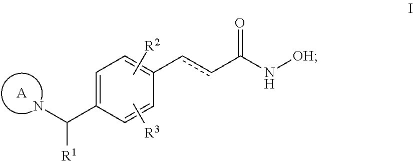

and pharmaceutically acceptable salts, hydrates, esters, and prodrugs thereof,
where: [symbol] is a) a single bond or b) a double bond;
ring A, including the nitrogen atom (N), is a 6-14 membered cycloheteroalkyl group optionally substituted with 1-5 R⁴ groups;
R¹ is a) H, b) a C₁₋₁₀ alkyl group, c) a C₂₋₁₀ alkenyl group, d) a C₂₋₁₀ alkynyl group, e) a C₃₋₁₄ cycloalkyl group, or f) a 3-14 membered cycloheteroalkyl group, wherein each of b)-f) optionally is substituted with 1-4 -L-R⁸ groups;
R² and R³ independently are a) H or b) halogen;
R⁴, at each occurrence, is a) halogen, b) oxo, c) ═NR⁶, d) ═CR⁶R⁷, e) OR⁶, f) —NR⁶R⁷, g) —C(O)R⁵, h) —C(O)OR⁶, i) —C(O)NR⁶R⁷, j) —S(O)ₘR⁵, k) a C₁₋₁₀ alkyl group, 1) a C₂₋₁₀ alkenyl group, m) a C₂₋₁₀ alkynyl group, n) a C₃₋₁₄ cycloalkyl group, o) a C₆₋₁₄ aryl group, p) a 3-14 membered cycloheteroalkyl group, or q) a 5-14 membered heteroaryl group, wherein each of k)-q) optionally is substituted with 1-4 groups independently selected from R⁸ and -L-R⁸, or
two R⁴ groups, taken together with the atom to which each R⁴ group is attached and any intervening ring atoms, form a) a C₃₋₁₄ cycloalkyl group, b) a C₆₋₁₄ aryl group, c) a 3-14 membered cycloheteroalkyl group, or d) a 5-14 membered heteroaryl group, wherein each of a)-d) optionally is substituted with 1-4 groups independently selected from R⁸ and -L-R⁸;
R⁵, at each occurrence, is a) H, b) a C₁₋₁₀ alkyl group, c) a C₂₋₁₀ alkenyl group, d) a C₂₋₁₀ alkynyl group, f) a C₃₋₁₄ cycloalkyl group, g) a C₆₋₁₄ aryl group, h) a 3-14 membered cycloheteroalkyl group, or i) a 5-14 membered heteroaryl group, wherein each of b)-i) optionally is substituted with 1-4 groups independently selected from R⁸ and -L-R⁸;
R⁶ and R⁷, at each occurrence, independently are a) H, b) —OR⁹, c) —NR⁹R¹⁰, d) —C(O)R⁹, e) —C(O)OR⁹, f) —C(O)NR⁹R¹⁰, g) —S(O)ₘR⁹, h) a C₁₋₁₀ alkyl group, i) a C₂₋₁₀ alkenyl group, j) a C₂₋₁₀ alkynyl group, k) a C₃₋₁₄ cycloalkyl group, 1) a C₆₋₁₄ aryl group, m) a 3-14 membered cycloheteroalkyl group, or n) a 5-14 membered heteroaryl group, wherein each of h)-n) optionally is substituted with 1-4 groups independently selected from R⁸ and -L-R⁸, or
R⁶ and R⁷, taken together with the atom to which each group is attached, form a) a C₃₋₁₄ cycloalkyl group, b) a C₆₋₁₄ aryl group, c) a 3-14 membered cycloheteroalkyl group, or d) a 5-14 membered heteroaryl group, each of which optionally is substituted with 1-4 groups independently selected from R⁸ and -L-R⁸;
R⁸, at each occurrence, is a) halogen, b) —CN, c) —NO₂, d) oxo, e) ═N-L-R⁹, f) —O-L-R⁹, g) —NR¹⁰-L-R⁹, h) —C(O)R⁹, i) —C(O)O-L-R⁹, j) —C(O)NR¹⁰-L-R⁹, k) —S(O)ₘR⁹, l) —Si(C₁₋₁₀ alkyl)₃, m) a C₁₋₁₀ alkyl group, n) a C₂₋₁₀ alkenyl group, o) a C₂₋₁₀ alkynyl group, p) a C₃₋₁₄ cycloalkyl group, q) a C₆₋₁₄ aryl group, r) a 3-14 membered cycloheteroalkyl group, or s) a 5-14 membered heteroaryl group, wherein each of the C₁₋₁₀ alkyl groups, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4 groups independently selected from R¹³ and -L-R¹³;
R⁹ and R¹⁰, at each occurrence, independently are a) H, b) —OR¹¹, c) —NR¹¹R¹², d) —C(O)R¹¹, e) —C(O)OR¹¹, f) —C(O)NR¹¹R¹², g) —S(O)ₘR¹¹, h) a C₁₋₁₀ alkyl group, i) a C₂₋₁₀ alkenyl group, j) a C₂₋₁₀ alkynyl group, k) a C₃₋₁₄ cycloalkyl group, l) a C₆₋₁₄ aryl group, m) a 3-14 membered cycloheteroalkyl group, or n) a 5-14 membered heteroaryl group, wherein each of h)-n) optionally is substituted with 1-4 groups independently selected from R¹³ and -L-R¹³;
R¹¹ and R¹², at each occurrence, independently are a) H, b) a C₁-C₁₀ alkyl group, c) a C₂₋₁₀ alkenyl group, d) a C₂₋₁₀ alkynyl group, e) a C₃₋₁₄ cycloalkyl group, f) a C₆₋₁₄ aryl group, g) a 3-14 membered cycloheteroalkyl group, or h) a 5-14 membered heteroaryl group, wherein each of b)-h) optionally is substituted with 1-4 groups independently selected from R¹³ and -L-R¹³;
R¹³, at each occurrence, is a) halogen, b) —CN, c) —NO₂, d) oxo, e) —OH, f) —NH₂, g) —NH(C₁₋₁₀ alkyl), h) —N(C₁₋₁₀ alkyl)₂, i) —CHO, j) —C(O)—C₁₋₁₀ alkyl, k) —C(O)OH, l) —C(O)—OC₁₋₁₀ alkyl, m) —C(O)SH, n) —C(O)—SC₁₋₁₀ alkyl, o) —C(O)NH₂, p) —C(O)NH(C₁₋₁₀ alkyl), q) —C(O)N(C₁₋₁₀ alkyl)₂, r) —C(S)H, s) —C(S)—C₁₋₁₀ alkyl, t) —C(S)NH₂, u) —C(S)NH(C₁₋₁₀ alkyl), v) —C(S)N(C₁₋₁₀ alkyl)₂, w) —C(NH)H, x) —C(NH)C₁₋₁₀ alkyl, y) —C(NH)NH₂, z) —C(NH)NH(C₁₋₁₀ alkyl), aa) —C(NH)N(C₁₋₁₀ alkyl)₂, ab) —C(NC₁₋₁₀ alkyl)H, ac)-C(NC₁₋₁₀ alkyl)-C₁₋₁₀ alkyl, ad) —C(NC₁₋₁₀ alkyl)NH(C₁₋₁₀ alkyl), ae) —C(NC₁₋₁₀ alkyl)N(C₁₋₁₀ alkyl)₂, af) —S(O)ₘH, ag) —S(O)ₘ—C₁₋₁₀ alkyl, ah) —S(O)₂OH, ai) —S(O)ₘ—OC₁₋₁₀ alkyl, aj) —S(O)ₘNH₂, ak) —S(O)ₘNH(C₁₋₁₀ alkyl), al) —S(O)ₘN(C₁₋₁₀ alkyl)₂, am) —Si(C₁₋₁₀ alkyl)₃, an) a C₁₋₁₀ alkyl group, ao) a C₂₋₁₀ alkenyl group, ap) a C₂₋₁₀ alkynyl group, aq) a C₁₋₁₀ alkoxy group, ar) a C₁₋₁₀ haloalkyl group, as) a C₃₋₁₄ cycloalkyl group, at) a C₆₋₁₄ aryl group, au) a 3-14 membered cycloheteroalkyl group, or av) a 5-14 membered heteroaryl group; L, at each occurrence, is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, e) a divalent C₁₋₁₀ alkoxy group, or f) a covalent bond; and
m, at each occurrence, is 0, 1, or 2.
In some embodiments, [symbol] can be a single bond. In some embodiments, [symbol] can be a double bond. For example, the double bond can be a cis-double bond (i.e., a Z-double bond) or a trans-double bond (i.e., an E-double bond). In certain embodiments, [symbol] can be a trans-double bond. Accordingly, compounds of the present teachings can have Formula Ia or Formula Ib:

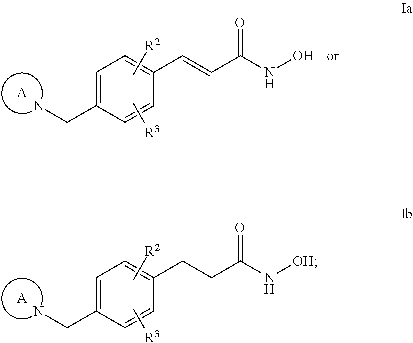

including pharmaceutically acceptable salts, hydrates, esters, and prodrugs thereof, where ring A, R², and R³ are as defined herein.

In various embodiments, ring A, including the nitrogen atom, can be a 6-12 membered cycloheteroalkyl group optionally substituted with 1-4 R⁴ groups, where R⁴ is as defined herein. For example, the 6-12 membered cycloheteroalkyl group can have 1, 2, 3, or 4 ring atoms independently selected from N, O, and S. In some embodiments, ring A, including the nitrogen atom, can be a six-membered cycloheteroalkyl group having 1-2 ring atoms independently selected from N, O, and S, where the six-membered cycloheteroalkyl group optionally can be substituted with 1-4 R⁴ groups and R⁴ is as defined herein. In certain embodiments, ring A can be selected from:

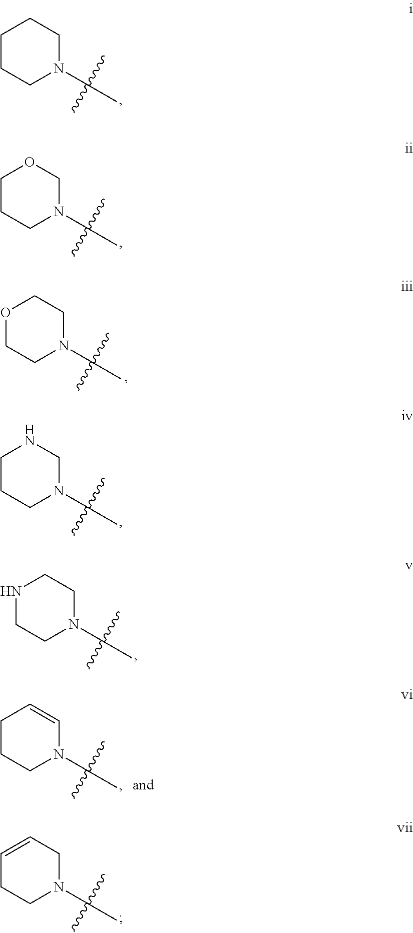

where each of i-vii optionally can be substituted with 1-4 R⁴ groups and R⁴ is as defined herein. In particular embodiments, ring A can be selected from i, iii, v, and vii, each of which optionally can be substituted with 1-4 R⁴ groups, where R⁴ is as defined herein.

In various embodiments, two R⁴ groups, taken together with the atom to which each R⁴ group is attached and any intervening ring atoms, can form a C₆₋₁₄ aryl group, a 3-14 membered cycloheteroalkyl group, or a 5-14 membered heteroaryl group, where each of the C₆₋₁₄ aryl group, the 3-14 membered cycloheteroalkyl group, and the 5-14 membered heteroaryl group optionally can be substituted with 1-4 groups independently selected from R⁸ and -L-R⁸, where L and R⁸ are as defined herein. In some embodiments, two R⁴ groups, taken together with the common atom to which the two R⁴ groups are attached and any intervening ring atoms, can form a C₆₋₁₄ aryl group, a 3-14 membered cycloheteroalkyl group, or a 5-14 membered heteroaryl group, where each of the C₆₋₁₄ aryl group, the 3-14 membered cycloheteroalkyl group, and the 5-14 membered heteroaryl group optionally can be substituted with 1-4 groups independently selected from R⁸ and -L-R⁸, where L and R⁸ are as defined herein. For example, the C₆₋₁₄ aryl group, the 3-14 membered cycloheteroalkyl group, or the 5-14 membered heteroaryl group can be selected from:

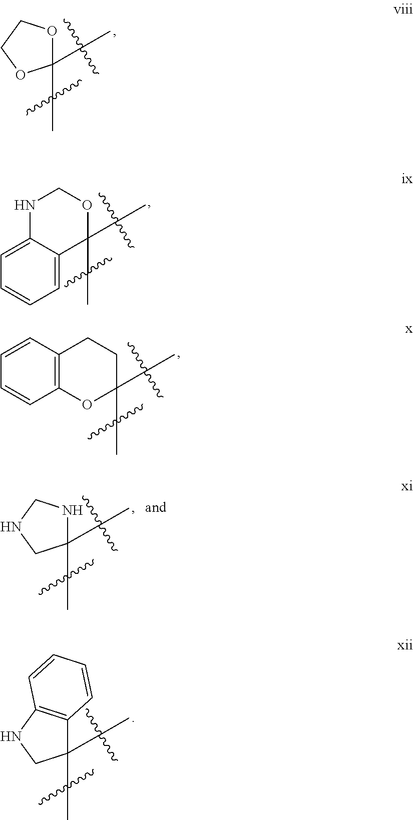

where each of viii-xii optionally can be substituted with 1-4 groups independently selected from R⁸ and -L-R⁸, where L and R⁸ are as defined herein. In some embodiments, two R⁴ groups, taken together with two atoms to which the two R⁴ groups respectively are attached and any intervening ring atoms, can form a C₆₋₁₄ aryl group, a 3-14 membered cycloheteroalkyl group, or a 5-14 membered heteroaryl group, where each of the C₆₋₁₄ aryl group, the 3-14 membered cycloheteroalkyl group, and the 5-14 membered heteroaryl group optionally can be substituted with 1-4 groups independently selected from R⁸ and -L-R⁸, where L and R⁸ are as defined herein. For example, the C₆₋₁₄ aryl group, the 3-14 membered cycloheteroalkyl group, or the 5-14 membered heteroaryl group can be selected from:

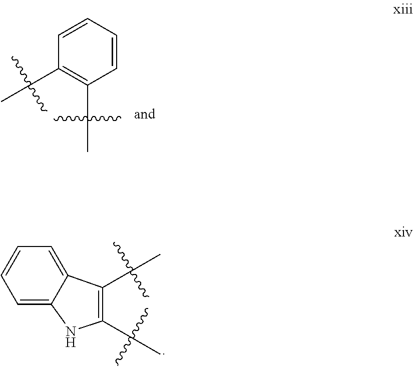

In various embodiments, ring A optionally can be substituted with 1-4 groups independently selected from oxo, ═NR⁶, ═CR⁶R⁷, —C(O)R⁵, —C(O)OR⁶, —C(O)NR⁶R⁷, —S(O)ₘR⁵, a C₁₋₁₀ alkyl group, a C₆₋₁₄ aryl group, a 3-14 membered cycloheteroalkyl group, and a 5-14 membered heteroaryl group, where each of the C₁₋₁₀ alkyl group, the C₆₋₁₄ aryl group, the 3-14 membered cycloheteroalkyl group, and the 5-14 membered heteroaryl group optionally can be substituted with 1-4 groups independently selected from R⁸ and -L-R⁸, where m, L, R⁵, R⁶, R⁷, and R⁸ are as defined herein. In some embodiments, ring A optionally can be substituted with oxo or ═N—OH. In some embodiment, ring A optionally can be substituted with ═CR⁶R⁷, where R⁶ and R⁷, taken together with the carbon atom to which they are attached, can form a C₃₋₁₄ cycloalkyl group, a C₆₋₁₄ aryl group, a 3-14 membered cycloheteroalkyl group, or a 5-14 membered heteroaryl group each optionally substituted with 1-4 groups independently selected from R⁸ and -L-R⁸, where L and R⁸ are as defined herein. In certain embodiments where ring A is substituted with ═CR⁶R⁷, R⁶ and R⁷, taken together with the carbon atom to which they are attached, can form a 5-14 membered heteroaryl group optionally substituted with 1-4 groups independently selected from R⁸ and -L-R⁸, where L and R⁸ are as defined herein. In particular embodiments, ring A optionally can be substituted with:

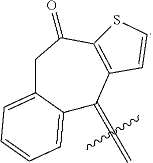

In some embodiments, ring A optionally can be substituted with 1-4 groups independently selected from —C(O)R⁵, —C(O)OR⁶, —C(O)NR⁶R⁷ and —SO₂R⁵, where R⁵, R⁶, and R⁷ are as defined herein. In certain embodiments, ring A optionally can be substituted with 1-4 groups independently selected from —C(O)R⁵, —C(O)OR⁶, —C(O)NR⁶R⁷ and —SO₂R⁵, where R⁵, R⁶, and R⁷, at each occurrence, independently can be a C₁₋₁₀ alkyl group, a C₃₋₁₄ cycloalkyl group, or a C₆₋₁₄ aryl group, where each of the C₁₋₁₀ alkyl group, the C₃₋₁₄ cycloalkyl group, and the C₆₋₁₄ aryl group optionally can be substituted with 1-4 -L-R⁸ groups, where L and R⁸ are as defined herein. In other embodiments, ring A optionally can be substituted with 1-4 groups independently selected from —C(O)R⁵, —C(O)OR⁶, —C(O)NR⁶R⁷ and —SO₂R⁵, where R⁵ and R⁶, at each occurrence, independently can be C₁₋₄ alkyl, C₃₋₆ cycloalkyl, benzyl, or phenyl optionally substituted with one methyl or fluoro group, and R⁷ is H.

In some embodiments, ring A optionally can be substituted with 1-4 groups independently selected from a C₁₋₁₀ alkyl group, a C₆₋₁₄ aryl group, a 3-14 membered cycloheteroalkyl group, and a 5-14 membered heteroaryl group, where each of the C₁₋₁₀ alkyl groups, the C₆₋₁₄ aryl group, the 3-14 membered cycloheteroalkyl group, and the 5-14 membered heteroaryl group optionally can be substituted with 1-4 groups independently selected from R⁸ and -L-R⁸, where L and R⁸ are as defined herein. For example, ring A optionally can be substituted with 1-4 groups independently selected from a methyl group, an ethyl group, a propyl group, and a benzyl group, each of which optionally can be substituted with 1-4 groups independently selected from R⁸ and -L-R⁸, where L and R⁸ are as defined herein. Moreover, ring A optionally can be substituted with 1 or 2 groups each independently selected from the group consisting of a methyl group, an ethyl group, a propyl group, a N(CH₃)C(O)CH₃ group, a CH₂C(O)NHOH group, a benzyl group, and a 4-bromobenzyl group.

In some embodiments, ring A optionally can be substituted with 1-4 groups independently selected from a C₆₋₁₄ aryl group, a 5-10 membered cycloheteroalkyl group, and a 5-10 membered heteroaryl group, each of which optionally can be substituted with 1-4 groups independently selected from R⁸ and -L-R⁸, where L and R⁸ are as defined herein. For example, the C₆₋₁₄ aryl group, the 5-10-membered cycloheteroalkyl group, or the 5-10 membered heteroaryl group can be selected from a phenyl group, an imidazolidyl group, an isoxazolyl group, an oxadiazolyl group, a pyridyl group, an indolyl group, an indolinyl group, a benzofuranyl group, a tetrahydroisoquinolyl group, a 1H-benzoimidazolyl group, and a benzopyridyl group. Moreover the phenyl group, imidazolidyl group, isoxazolyl group, oxadiazolyl group, pyridyl group, indolyl group, indolinyl group, benzofuranyl group, tetrahydroisoquinolyl group, 1H-benzoimidazolyl group, and benzopyridyl group can be substituted with one or two substituents each independently selected from the group consisting of C₁₋₄ alkyl optionally substituted by one hydroxy group; isobutenyl, C₃₋₆ cycloalkyl, phenyl, morpholinyl, oxo, C(O)C₆H₅, —C(O)morpholinyl, —C(O)NHOH, —COOCH₂CH₃, halo, methoxy, and 2-oxo-pyrrolidin-1-ylmethyl.

In various embodiments, R² and R³ can independently be selected from H, F, Cl, and Br. In some embodiments, R² and R³ can independently be selected from H, F, and Cl. In some embodiments R² and R³ are both hydrogen. In other embodiments R² is hydrogen and R³ is fluoro or chloro.

In various embodiments, R¹ can be H, a C₁₋₁₀ alkyl group, a C₃₋₁₄ cycloalkyl group, or a 3-14 membered cycloheteroalkyl group, where each of the C₁₋₁₀ alkyl groups, the C₃₋₁₄ cycloalkyl group, and the 3-14 membered cycloheteroalkyl group optionally can be substituted with 1-4 -L-R⁸ groups, where L and R⁸ are as defined herein. In certain embodiments, R¹ can be H, a C₁₋₁₀ alkyl group, or a C₃₋₁₄ cycloalkyl group, where each of the C₁₋₁₀ alkyl group and a C₃₋₁₄ cycloalkyl group optionally can be substituted with 1-4 -L-R⁸ groups, where L and R⁸ are as defined herein. In particular embodiments, R¹ can be H. In particular embodiments, R¹ can be a methyl group, an ethyl group, an isopropyl group, a t-butyl group, a cyclopropyl group, a cyclopentyl group, or a cyclohexyl group, each optionally substituted with 1-4 groups independently selected from halogen.

Compounds of the present teachings can be selected from the compounds in Table 1.

TABLE 1

| # | Structure | Name |
|---|---|---|
| 1 | 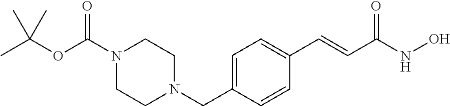 | 4-[4-((E)-2-Hydroxycarbamoyl- vinyl)-benzyl]-piperazine-1- carboxylic acid tert-butyl ester |
| 2 | 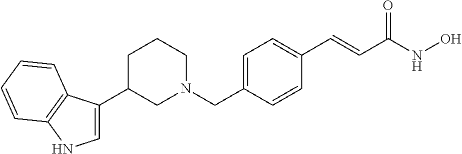 | (E)-N-Hydroxy-3-{4-[3-(1H-indol-3- yl)-piperidin-1-ylmethyl]-phenyl}- acrylamide |
| 3 | 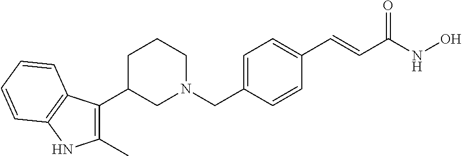 | (E)-N-Hydroxy-3-{4-[(R)-3-(2- methyl-1H-indol-3-yl)-piperidin-1- ylmethyl]-phenyl}-acrylamide |
| 4 | 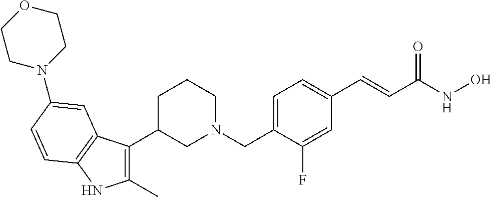 | (E)-3-{3-Fluoro-4-[3-(2-methyl-5- morpholin-4-yl-1H-indol-3-yl)- piperidin-1-ylmethyl]-phenyl}-N- hydroxy-acrylamide |
| 5 | 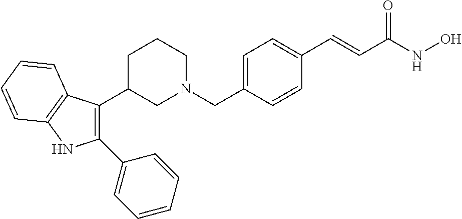 | (E)-N-Hydroxy-3-{4-[3-(2-phenyl- 1H-indol-3-yl)-piperidin-1-ylmethyl]- phenyl}-acrylamide |
| 6 | 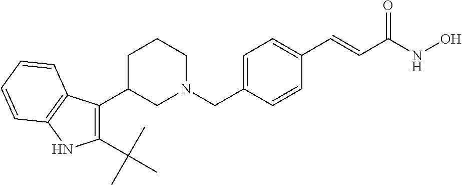 | (E)-3-{4-[3-(2-tert-Butyl-1H-indol-3- yl)-piperidin-1-ylmethyl]-phenyl}-N- hydroxy-acrylamide |
| 7 | 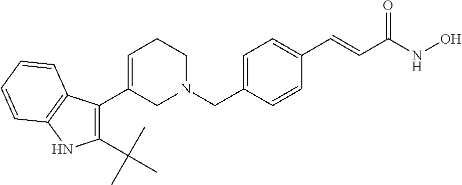 | (E)-3-{4-[5-(2-tert-Butyl-1H-indol-3- yl)-3,6-dihydro-2H-pyridin-1- ylmethyl]-phenyl}-N-hydroxy- acrylamide |
| 8 | 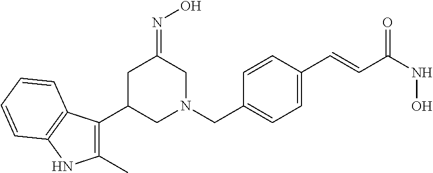 | (E)-N-Hydroxy-3-{4-[3-[(Z)- hydroxyimino]-5-(2-methyl-1H-indol- 3-yl)-piperidin-1-ylmethyl]-phenyl}- acrylamide |
| 9 | 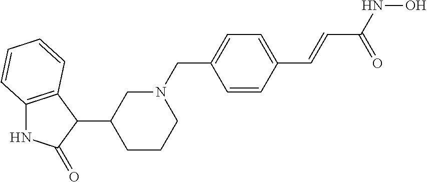 | (E)-N-Hydroxy-3-{4-[3-(2-oxo-2,3- dihydro-1H-indol-3-yl)-piperidin-1- ylmethyl]-phenyl}-acrylamide |
| 10 | 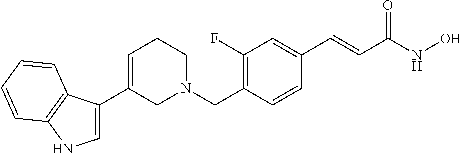 | (E)-3-{3-Fluoro-4-[3-(1H-indol-3-yl)- piperidin-1-ylmethyl]-phenyl}-N- hydroxy-acrylamide |
| 11 | 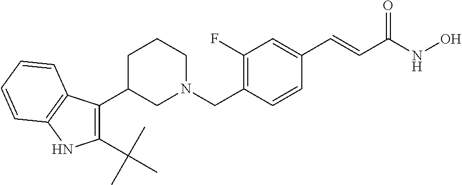 | (E)-3-{4-[(S)-3-(2-tert-Butyl-1H- indol-3-yl)-piperidin-1-ylmethyl]-3- fluoro-phenyl}-N-hydroxy-acrylamide |
| 12 | 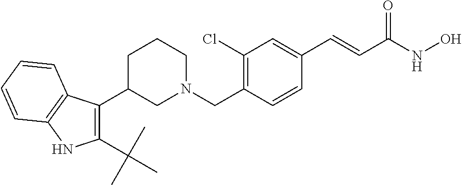 | (E)-3-{4-[3-(2-tert-Butyl-1H-indol-3- yl)-piperidin-1-ylmethyl]-3-chloro- phenyl}-N-hydroxy-acrylamide |
| 13 | 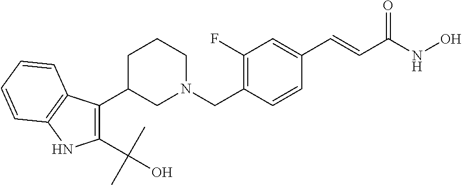 | (E)-3-(3-Fluoro-4-{3-[2-(1-hydroxy-1- methyl-ethyl)-1H-indol-3-yl]- piperidin-1-ylmethyl}-phenyl)-N- hydroxy-acrylamide |
| 14 | 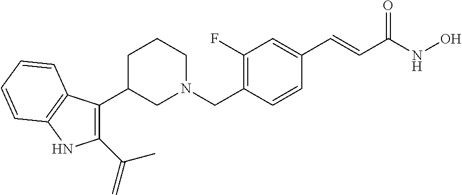 | (E)-3-{3-Fluoro-4-[3-(2-isopropenyl- 1H-indol-3-yl)-piperidin-1-ylmethyl]- phenyl}-N-hydroxy-acrylamide |
| 15 | 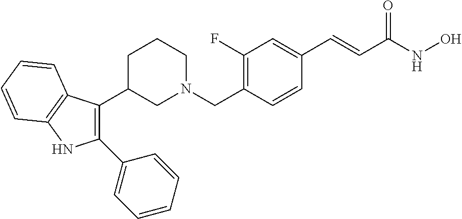 | (E)-3-{3-Fluoro-4-[3-(2-phenyl-1H- indol-3-yl)-piperidin-1-ylmethyl]- phenyl}-N-hydroxy-acrylamide |
| 16 | 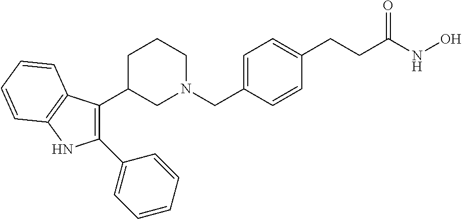 | N-Hydroxy-3-{4-[3-(2-phenyl-1H- indol-3-yl)-piperidin-1-ylmethyl]- phenyl}-propionamide |
| 17 | 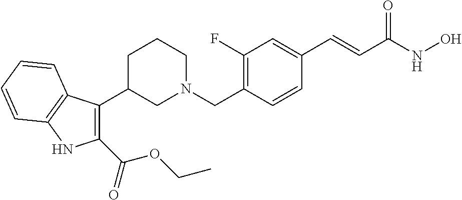 | 3-{1-[2-Fluoro-4-((E)-2- hydroxycarbamoyl-vinyl)-benzyl]- piperidin-3-yl}-1H-indole-2- carboxylic acid ethyl ester |
| 18 | 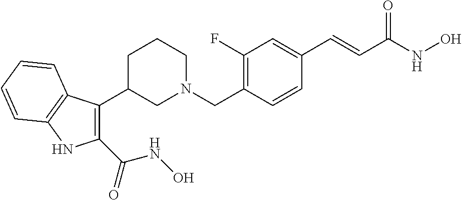 | 3-{1-[2-Fluoro-4-((E)-2- hydroxycarbamoyl-vinyl)-benzyl]- piperidin-3-yl}-1H-indole-2- carboxylic acid hydroxyamide |
| 19 | 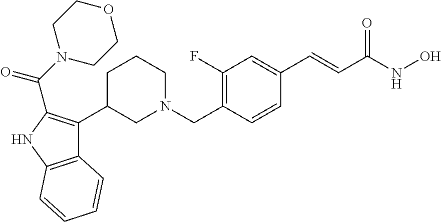 | (E)-3-(3-Fluoro-4-{3-[2-(morpholine- 4-carbonyl)-1H-indol-3-yl]-piperidin- 1-ylmethyl}-phenyl)-N-hydroxy- acrylamide |
| 20 | 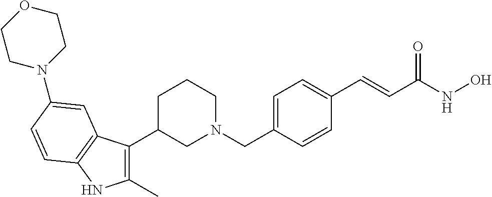 | (E)-N-Hydroxy-3-{4-[3-(2-methyl-5- morpholin-4-yl-1H-indol-3-yl)- piperidin-1-ylmethyl]-phenyl}- acrylamide |
| 21 | 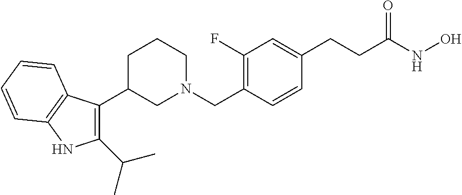 | 3-{3-Fluoro-4-[3-(2-isopropyl-1H- indol-3-yl)-piperidin-1-ylmethyl]- phenyl}-N-hydroxy-propionamide |
| 22 | 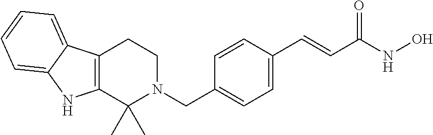 | (E)-3-[4-(1,1-Dimethyl-1,3,4,9- tetrahydro-beta-carbolin-2-ylmethyl)- phenyl]-N-hydroxy-acrylamide |
| 23 | 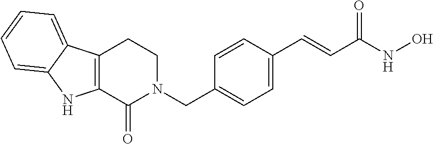 | (E)-N-Hydroxy-3-[4-(1-oxo-1,3,4,9- tetrahydro-beta-carbolin-2-ylmethyl)- phenyl]-acrylamide |
| 24 | 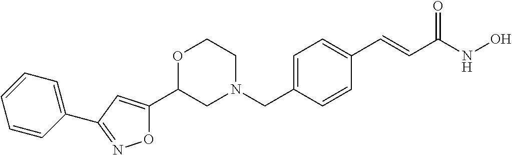 | (E)-N-Hydroxy-3-{4-[2-(3-phenyl- isoxazol-5-yl)-morpholin-4-ylmethyl]- phenyl}-acrylamide |
| 25 | 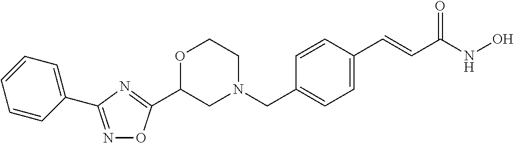 | (E)-N-Hydroxy-3-{4-[2-(3-phenyl- [1,2,4]oxadiazol-5-yl)-morpholin-4- ylmethyl]-phenyl}-acrylamide |
| 26 | 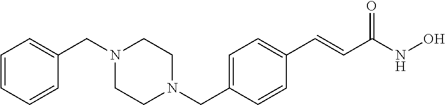 | (E)-3-[4-(4-Benzyl-piperazin-1- ylmethyl)-phenyl]-N-hydroxy- acrylamide |
| 27 | 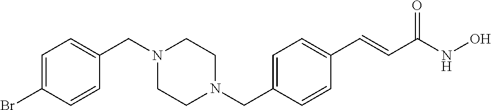 | (E)-3-{4-[4-(4-Bromo-benzyl)- piperazin-1-ylmethyl]-phenyl}-N- hydroxy-acrylamide |
| 28 | 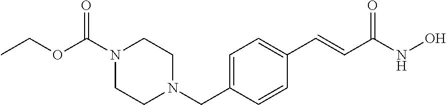 | 4-[4-((E)-2-Hydroxycarbamoyl- vinyl)-benzyl]-piperazine-1- carboxylic acid ethyl ester |
| 29 | 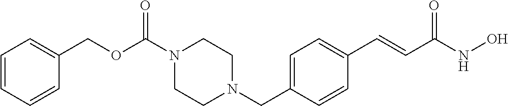 | 4-[4-((E)-2-Hydroxycarbamoyl- vinyl)-benzyl]-piperazine-1- carboxylic acid benzyl ester |
| 30 | 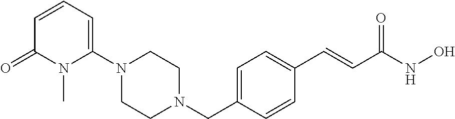 | (E)-N-Hydroxy-3-{4-[4-(1-methyl-6- oxo-1,6-dihydro-pyridin-2-yl)- piperazin-1-ylmethyl]-phenyl}- acrylamide |
| 31 | 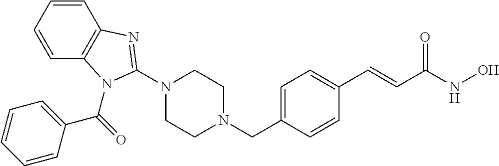 | (E)-3-{4-[4-(1-Benzoyl-1H- benzoimidazol-2-yl)-piperazin-1- ylmethyl]-phenyl}-N-hydroxy- acrylamide |
| 32 | 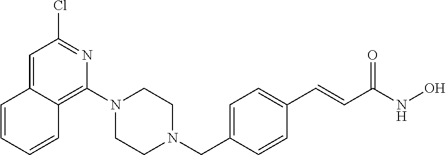 | (E)-3-{4-[4-(3-Chloro-isoquinolin-1- yl)-piperazin-1-ylmethyl]-phenyl}-N- hydroxy-acrylamide |
| 33 | 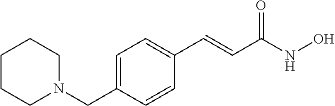 | (E)-N-Hydroxy-3-(4-piperidin-1- ylmethyl-phenyl)-acrylamide |
| 34 | 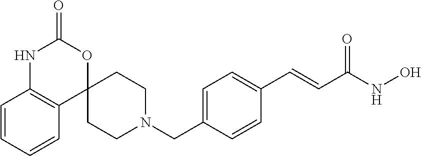 | (E)-3-Spiro[4H-3,1-benzoxazine-4,4′- peperidin]-2(1H)-1-ylmethyl)- phenyl]-N-hydroxy-acrylamide |
| 35 | 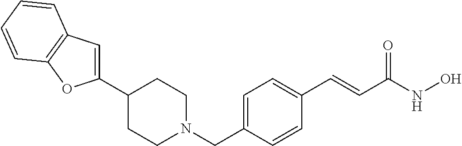 | (E)-3-[4-(4-Benzofuran-2-yl- piperidin-1-ylmethyl)-phenyl]-N- hydroxy-acrylamide |
| 36 | 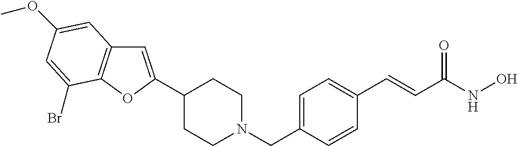 | (E)-3-{4-[4-(7-Bromo-5-methoxy- benzofuran-2-yl)-piperidin-1- ylmethyl]-phenyl}-N-hydroxy- acrylamide |
| 37 | 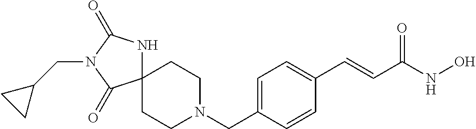 | (E)-3-[4-(3-Cyclopropylmethyl-2,4- dioxo-1,3,8-triaza-spiro[4.5]dec-8- ylmethyl)-phenyl]-N-hydroxy- acrylamide |
| 38 | 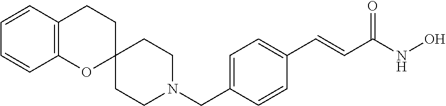 | (E)-N-Hydroxy-3-(4-(spiro[chroman- 2,4′-piperidine]-1′- ylmethyl)phenyl)acrylamide |
| 39 | 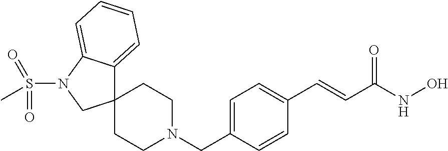 | (E)-N-Hydroxy-3-(4-((1- methylsulfonyl)spiro[indoline-3,4′- piperidine]-1′- yl)methyl)phenyl)acrylamide |
| 40 | 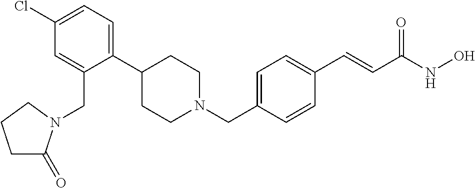 | (E)-3-(4-{4-[4-Chloro-2-(2-oxo- pyrrolidin-1-ylmethyl)-phenyl]- piperidin-1-ylmethyl}-phenyl)-N- hydroxy-acrylamide |
| 41 | 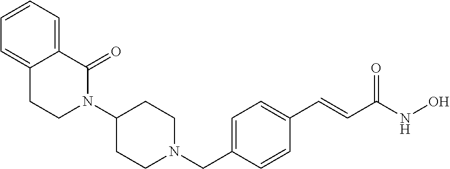 | (E)-N-Hydroxy-3-{4-[4-(1-oxo-3,4- dihydro-1H-isoquinolin-2-yl)- piperidin-1-ylmethyl]-phenyl}- acrylamide |
| 42 | 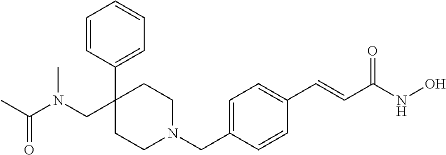 | (E)-3-(4-{4-[(Acetyl-methyl-amino)- methyl]-4-phenyl-piperidin-1- ylmethyl}-phenyl)-N-hydroxy- acrylamide |
| 43 | 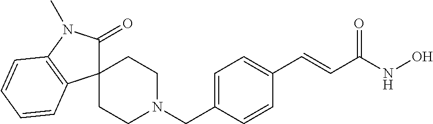 | (E)-N-Hydroxy-3-(4-((1-methyl-2- oxospiro[indoline-3,4′-piperidine]-1′- yl)methyl)phenyl)acrylamide |
| 44 | 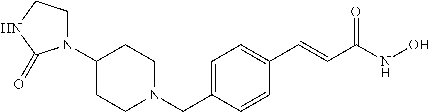 | (E)-N-Hydroxy-3-{4-[4-(2-oxo- imidazolidin-1-yl)-piperidin-1- ylmethyl]-phenyl}-acrylamide |
| 45 | 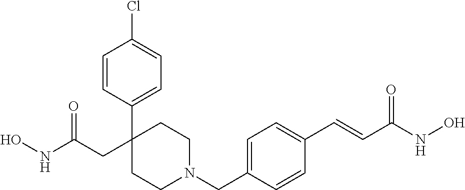 | (E)-3-{4-[4-(4-Chloro-phenyl)-4- hydroxycarbamoylmethyl-piperidin-1- ylmethyl]-phenyl}-N-hydroxy- acrylamide |
| 46 | 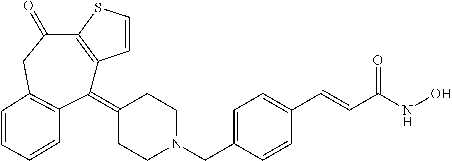 | (E)-N-Hydroxy-3-{4-[4-(10-oxo-9,10- dihydro-1-thia-benzo[f]azulen-4- ylidene)-piperidin-1-ylmethyl]- phenyl}-acrylamide |
| 47 | 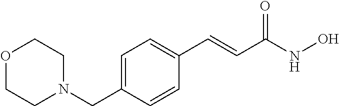 | (E)-N-Hydroxy-3-(4-morpholin-4- ylmethyl-phenyl)-acrylamide |
| 48 | 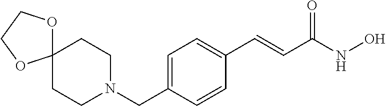 | (E)-3-[4-(1,4-Dioxa-8-aza- spiro[4.5]dec-8-ylmethyl)-phenyl]-N- hydroxy-acrylamide |
| 49 | 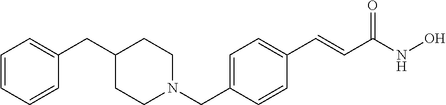 | (E)-3-[4-(4-Benzyl-piperidin-1- ylmethyl)-phenyl]-N-hydroxy- acrylamide |
| 50 | 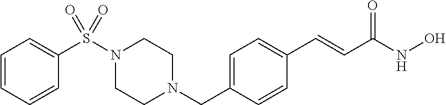 | (E)-3-[4-(4-Benzenesulfonyl- piperazin-1-ylmethyl)-phenyl]-N- hydroxy-acrylamide |
| 51 | 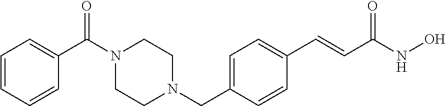 | (E)-3-[4-(4-Benzoyl-piperazin-1- ylmethyl)-phenyl]-N-hydroxy- acrylamide |
| 52 | 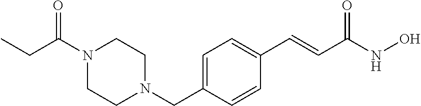 | (E)-N-Hydroxy-3-[4-(4-propionyl- piperazin-l-ylmethyl)-phenyl]- acrylamide |
| 53 | 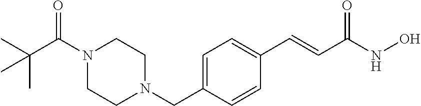 | (E)-3-{4-[4-(3,3-Dimethyl-butyryl)- piperazin-1-ylmethyl]-phenyl}-N- hydroxy-acrylamide |
| 54 | 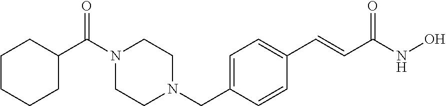 | (E)-3-[4-(4-Cyclohexanecarbonyl- piperazin-1-ylmethyl)-phenyl]-N- hydroxy-acrylamide |
| 55 | 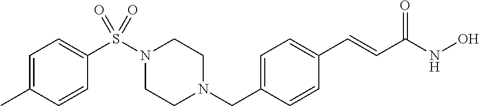 | (E)-N-Hydroxy-3-{4-[4-(toluene-4- sulfonyl)-piperazin-1-ylmethyl]- phenyl}-acrylamide |
| 56 | 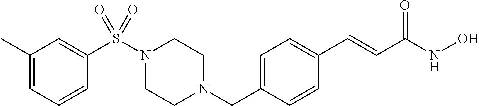 | (E)-N-Hydroxy-3-{4-[4-(toluene-3- sulfonyl)-piperazin-1-ylmethyl]- phenyl}-acrylamide |
| 57 | 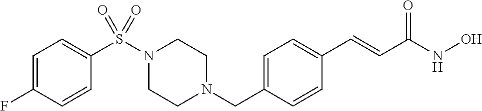 | (E)-3-{4-[4-(4-Fluoro- benzenesulfonyl)-piperazin-1- ylmethyl]-phenyl}-N-hydroxy- acrylamide |
| 58 | 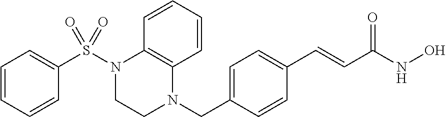 | (E)-3-[4-(4-Benzenesulfonyl-3,4- dihydro-2H-quinoxalin-1-ylmethyl)- phenyl]-N-hydroxy-acrylamide |
| 59 | 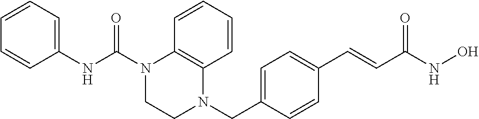 | 4-[4-((E)-2-Hydroxycarbamoyl- vinyl)-benzyl]-3,4-dihydro-2H- quinoxaline-1-carboxylic acid phenylamide |
| 60 | 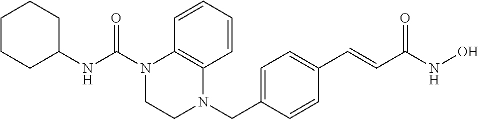 | (E)-3-[4-(4-Cyclohexanecarbonyl-3,4- dihydro-2H-quinoxalin-1-ylmethyl)- phenyl]-N-hydroxy-acrylamide |
| 61 | 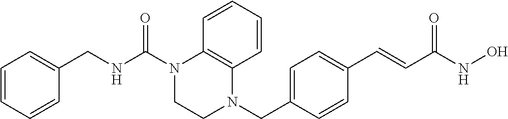 | 4-[4-((E)-2-Hydroxycarbamoyl- vinyl)-benzyl]-3,4-dihydro-2H- quinoxaline-l-carboxylic acid benzylamide |

Also provided in accordance with the present teachings are prodrugs of the compounds disclosed herein. As used herein, “prodrug” refers to a compound (“parent compound”) having a moiety that produces, generates, or releases a compound of the present teachings (“active compound”) when administered to a mammalian subject. Prodrugs can be prepared by modifying functional groups present in the active compounds in such a way that the modifications can be removed, either by routine manipulation or in vivo, from the parent compounds. Examples of prodrugs include compounds that contain one or more molecular moieties that are appended to a hydroxyl, amino, sulfhydryl, or carboxyl group of the active compounds, and that, when administered to a mammalian subject, is/are cleaved in vivo to form the free hydroxyl, amino, sulfhydryl, or carboxyl group, respectively, and to release the active compound. Examples of prodrugs can include acetate, formate, and benzoate derivatives of hydroxy and amino functional groups in the compounds of the present teachings. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, the entire disclosures of which are incorporated by reference herein for all purposes.

Ester forms of the compounds according to the present teachings include pharmaceutically acceptable esters known in the art that can be metabolized into the free acid form, such as a free carboxylic acid form, in a mammal body. Examples of such esters include alkyl esters (e.g., alkyls of 1 to 10 carbon atoms), cycloalkyl esters (e.g., cycloalkyls of 3-10 carbon atoms), aryl esters (e.g., aryls of 6-14 carbon atoms, including of 6-10 carbon atoms), and heterocyclic analogues thereof (e.g., heterocyclics of 3-14 ring atoms, 1-3 of which can be selected from O, N, and S) and the alcoholic residue can carry further substituents. In some embodiments, esters of the compounds disclosed herein can be C₁₋₁₀ alkyl esters, such as methyl esters, ethyl esters, propyl esters, isopropyl esters, butyl esters, isobutyl esters, t-butyl esters, pentyl esters, isopentyl esters, neopentyl esters, hexyl esters, cyclopropylmethyl esters, and benzyl esters, C₃₋₁₀ cycloalkyl esters, such as cyclopropyl esters, cyclobutyl esters, cyclopentyl esters, and cyclohexyl esters, or aryl esters, such as phenyl esters and tolyl ester.

Pharmaceutically acceptable salts of compounds of the present teachings, which can have an acidic moiety, can be formed using organic or inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di-, or tri-lower alkylamine (e.g., ethyl-tert-butylamine, diethylamine, diisopropylamine, triethylamine, tributylamine, or dimethylpropylamine), or a mono-, di-, or trihydroxy lower alkylamine (e.g., mono-, di- or triethanolamine). Non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from any of the following acids: acetic, benzenesulfonic, benzoic, camphorsulfonic, citric, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, as well as other known pharmaceutically acceptable acids.

In another aspect, the present teachings provide pharmaceutical compositions including at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington: The Science and Practice of Pharmacy, 20th edition, Alfonoso R. Gennaro (ed.), Lippincott Williams & Wilkins, Baltimore, Md. (2000), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be useful for inhibiting a deacetylase in a cell. Accordingly, another aspect of the present teachings includes a method of contacting a cell with one or more compounds of the present teachings (or a salt, hydrate, ester, or prodrug thereof) or a composition that includes one or more compounds of the present teachings. In certain embodiments, the composition can further include one or more pharmaceutically acceptable carrier or excipients.

Compounds of the present teachings can be useful for the treatment, inhibition, prevention, or diagnosis of a pathological condition or disorder in a mammal, for example, a human. Accordingly, another aspect of the present teachings includes a method of providing to a mammal a compound of the present teachings (or its pharmaceutically acceptable salt, hydrate, ester, or prodrug) or a pharmaceutical composition that includes one or more compounds of the present teachings in combination or association with a pharmaceutically acceptable carrier. Compounds of the present teachings can be administered alone or in combination with other therapeutically effective compounds or therapies for the treatment, inhibition, prevention, or diagnosis of the pathological condition or disorder. As used herein, “therapeutically effective” refers to a substance or an amount that elicits a desirable biological activity or effect.

In various embodiments, the present teachings can further include use of the compounds disclosed herein as active therapeutic substances for the treatment or inhibition of a pathological condition or disorder, for example, a condition mediated wholly or in part by one or more deacetylases, such as an undesired proliferative condition; a neurodegenerative disease, including Alzheimer's disease, Hungtington's disease, Rubenstein-Taybis syndrome, Parkinson's disease, muscular dystrophy, spinal muscular atrophy, Rett's syndrome, and the like; a cardiovascular disease, including heart failure, cardiac hypertrophy, thrombosis, and the like; an autoimmune disease, including Lupus, atherosclerosis, scleroderma, and the like; an inflammatory disorder, including arthritis and arthritic conditions (e.g., osteoarthritis, rheumatoid arthritis, and the like), and other chronic inflammatory disorders (e.g., chronic asthma, arterial or post-transplantational atherosclerosis, endometriosis, and the like); an undesired immunological process; stroke; and an fungal infection. In some embodiments, the undesired proliferative condition includes a cancer (e.g., brain cancer, kidney cancer, liver cancer, adrenal gland cancer, bladder cancer, breast tumor, stomach cancer including gastric tumors, esophagus cancer, ovarian cancer, colon cancer, rectum cancer, prostate cancer, pancrea cancer, lung cancer including small cell lung cancer, vagina cancer, thyroid cancer, sarcoma, glioblastomas, multiple myeloma, gastrointestinal cancer, lung cancer, colon cancer, breast cancer, ovarian cancer, bladder cancer), a tumor, a fibrosis, and the like; a neoplasia, including mammary carcinoma, leukemia, and the like; and an epidermal hyperproliferation, including psoriasis, prostate hyperplasia, and the like. In certain embodiments, the present teachings can provide methods of treating these pathological conditions and disorders using the compounds described herein. As used herein, “treating” refers to partially or completely alleviating and/or ameliorating the condition or symptoms thereof. In particular embodiments, the methods can include identifying a mammal having a pathological condition or disorder mediated by deacetylases, and providing to the mammal a therapeutically effective amount of a compound as described herein. In some embodiments, the method can include administering to a mammal a pharmaceutical composition that can include a compound disclosed herein in combination or association with a pharmaceutically acceptable carrier.

In various embodiments, the present teachings can further include use of the compounds disclosed herein as active therapeutic substances for the prevention of a pathological condition or disorder, for example, a condition mediated wholly or in part by one or more deacetylases, such as an undesired proliferative condition; a neurodegenerative disease, including Alzheimer's disease, Hungtington's disease, Rubenstein-Taybis syndrome, Parkinson's disease, muscular dystrophy, spinal muscular atrophy, Rett's syndrome, and the like; a cardiovascular disease, including heart failure, cardiac hypertrophy, thrombosis, and the like; an autoimmune disease, including Lupus, atherosclerosis, scleroderma, and the like; an inflammatory disorder, including arthritis and arthritic conditions (e.g., osteoarthritis, rheumatoid arthritis, and the like), and other chronic inflammatory disorders (e.g., chronic asthma, arterial or post-transplantational atherosclerosis, endometriosis, and the like); an undesired immunological process; stroke; and an fungal infection. In some embodiments, the undesired proliferative condition includes a cancer (e.g., brain cancer, kidney cancer, liver cancer, adrenal gland cancer, bladder cancer, breast tumor, stomach cancer including gastric tumors, esophagus cancer, ovarian cancer, colon cancer, rectum cancer, prostate cancer, pancrea cancer, lung cancer including small cell lung cancer, vagina cancer, thyroid cancer, sarcoma, glioblastomas, multiple myeloma, gastrointestinal cancer, lung cancer, colon cancer, breast cancer, ovarian cancer, bladder cancer), a tumor, a fibrosis, and the like; a neoplasia, including mammary carcinoma, leukemia, and the like; and an epidermal hyperproliferation, including psoriasis, prostate hyperplasia, and the like. In some embodiments, the present teachings can provide methods of preventing these pathological conditions and disorders using the compounds described herein. In certain embodiments, the methods can include identifying a mammal that could potentially have a pathological condition or disorder mediated by deacetylases, and providing to the mammal a therapeutically effective amount of a compound as described herein. In some embodiments, the method can include administering to a mammal a pharmaceutical composition that can include a compound disclosed herein in combination or association with a pharmaceutically acceptable carrier.

Cardiac hypertrophy in response to an increased workload imposed on the heart is a fundamental adaptive mechanism. It is a specialized process reflecting a quantitative increase in cell size and mass (rather than cell number) as the result of any or a combination of neural, endocrine or mechanical stimuli. Hypertension, another factor involved in cardiac hypertrophy, is a frequent precursor of congestive heart failure. When heart failure occurs, the left ventricle usually is hypertrophied and dilated and indices of systolic function, such as ejection fraction, are reduced. Clearly, the cardiac hypertrophic response is a complex syndrome and the elucidation of the pathways leading to cardiac hypertrophy will be beneficial in the treatment of heart disease resulting from a various stimuli.

In an embodiment, there is provided a method of preventing pathologic cardiac hypertrophy and heart failure with the compounds of the present invention. The method includes administering to the patient a histone deacetylase inhibitor. Administration may comprise intravenous, oral, transdermal, sustained release, suppository, or sublingual administration. The patient at risk may exhibit one or more of long standing uncontrolled hypertension, uncorrected valvular disease, chronic angina and/or recent myocardial infarction.

In one embodiment of the present invention, methods for the treatment of cardiac hypertrophy utilizing HDAC inhibitors are provided. For the purposes of the present application, treatment comprises reducing one or more of the symptoms of cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. In addition, use of HDAC inhibitors may prevent cardiac hypertrophy and its associated symptoms from arising.

Treatment regimens would vary depending on the clinical situation. However, long term maintenance would appear to be appropriate in most circumstances. It also may be desirable treat hypertrophy with HDAC inhibitors intermittently, such as within brief window during disease progression. At present, testing indicates that the optimal dosage for an HDAC inhibitor will be the maximal dose before significant toxicity occurs.

In another embodiment, it is envisioned to use an HDAC inhibition in combination with other therapeutic modalities. Thus, in addition to the therapies described above, one may also provide to the patient more “standard” pharmaceutical cardiac therapies. Examples of standard therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors.

In an embodiment, the cardiovascular indications for which the HDAC inhibitors may be used include: diastolic dysfunction, myocardial Infarction (systolic dysfunction), inhibition of overall cardiac remodeling in both acute and chronic heart failure conditions, adriamycin induced cardiotoxicity, inducing cardioprotection from ischemic events, and for the use of hemorrhagic shock and resuscitation.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders, tablet-disintegrating agents, or encapsulating materials. The compounds can be formulated in conventional manner, for example, in a manner similar to that used for known HDAC inhibitors. Oral formulations containing an active compound disclosed herein can include any conventionally used oral form, including tablets, capsules, buccal forms, troches, lozenges and oral liquids, suspensions, and solutions. In powders, the carrier can be a finely divided solid, which is an admixture with a finely divided active compound. In tablets, an active compound can be mixed with a carrier having the necessary compression properties in suitable proportions and compacted in the shape and size desired. The powders and tablets may contain up to 99% of the active compound.

Capsules can contain mixtures of active compound(s) optionally with inert filler(s) and/or diluent(s) such as the pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending agents, or stabilizing agents, including magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Preferred surface modifying agents include nonionic and anionic surface modifying agents. Representative examples of surface modifying agents include poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the active compound(s). The oral formulation can also consist of administering an active compound in water or fruit juice, containing appropriate solubilizers or emulsifiers as needed.

Liquid carriers can be used in preparing solutions, suspensions, emulsions, syrups, and elixirs. An active compound described herein can be dissolved or suspended in a pharmaceutically acceptable liquid carrier such as water, an organic solvent, a mixture thereof, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include water (particularly containing additives as described above, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal, or subcutaneous injection. Sterile solutions can also be administered intravenously. Compositions for oral administration can be in either liquid or solid form.

The pharmaceutical composition can be in unit dosage form, for example, as tablets, capsules, powders, solutions, suspensions, emulsions, granules, or suppositories. In such form, the pharmaceutical composition can be sub-divided in unit dose(s) containing appropriate quantities of the active compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form may contain from about 1 mg/kg of active compound to about 500 mg/kg of active compound, and can be given in a single dose or in two or more doses. Such doses can be administered in any manner useful in directing the active compound(s) to the recipient's bloodstream, including orally, via implants, parenterally (including intravenous, intraperitoneal and subcutaneous injections), rectally, vaginally, and transdermally. Such administrations can be carried out using the compounds of the present teachings including pharmaceutically acceptable salts, hydrates, and esters thereof, in lotions, creams, foams, patches, suspensions, solutions, and suppositories (rectal and vaginal).

When administered for the treatment or inhibition of a particular pathologic condition or disorder, it is understood that an effective dosage can vary depending upon the particular compound utilized, the mode of administration, and/or severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. In preventive applications, a compound of the present teachings can be provided to a patient that can suffer from a disease in an amount sufficient to prevent or at least delay the symptoms of the disease and its complications. The dosage to be used in the treatment of a specific individual typically must be subjectively determined by the attending physician. The variables involved include the specific condition and its state as well as the size, age and response pattern of the patient.

In some cases, for example, those in which the lung is the targeted organ, it may be desirable to administer a compound directly to the airways of the patient, using devices such as metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these active compounds or pharmaceutically acceptable salts, hydrates, or esters thereof can be prepared in water mixed with a suitable surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In preferred embodiments, the form is sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds of the present teachings can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal). Topical formulations that deliver active compound(s) through the epidermis can be useful for localized treatment of a pathologic condition or disorder.

Transdermal administration can be accomplished through the use of a transdermal patch containing an active compound and a carrier that can be inert to the active compound, can be non-toxic to the skin, and can allow delivery of the active compound for systemic absorption into the blood stream via the skin. The carrier can take any number of forms such as creams, ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active compound can also be suitable. A variety of occlusive devices can be used to release the active compound into the blood stream, such as a semi-permeable membrane covering a reservoir containing the active compound with or without a carrier, or a matrix containing the active compound. Other occlusive devices known in the literature are also contemplated.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can also be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound disclosed herein with other agents effective in the treatment of the target disease. For proliferative diseases, other active compounds (i.e., other active ingredients or agents) effective in their treatment, and particularly in the treatment of cancers and tumors, can be administered with active compounds of the present teachings. The other agents can be administered at the same time or at different times than the compounds disclosed herein.

The compounds of the present teachings can be prepared in accordance with the procedures outlined in the scheme below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the preparation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC), gas chromatograph (GC), or thin layer chromatography.

Preparation of Compounds can Involve the Protection and Deprotection of Various chemical groups. The need for protection and deprotection and the selection of appropriate protecting groups can be readily determined by one skilled in the art. The chemistry of protecting groups can be found, for example, in Greene, et al., Protective Groups in Organic Synthesis, 4th Ed., Wiley & Sons, 2006, the entire disclosure of which is incorporated by reference herein for all purposes.

The reactions described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

### Example 1

### Preparation of 4-[4-((E)-2-methoxycarbonyl-vinyl)-benzyl]-piperazine-1-carboxylic acid tert-butyl ester

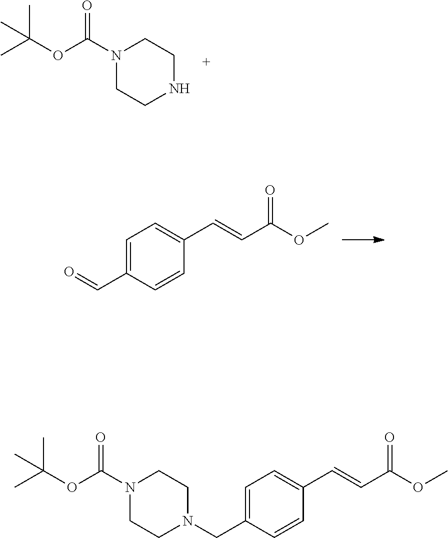

To a well-stirred solution of piperazine-1-carboxylic acid tert-butyl ester (6 g, 10 mmol) and (E)-3-(4-formyl-phenyl)-acrylic acid methyl ester hydrochloride (7.42 g, 10 mmol) in tetrahydrofuran (150 mL) is added acetic acid (2.25 mL, 39 mmol) and the resulting solution is stirred for 15 minutes. Sodium triacetoxyborohydride (8.76 g, 39 mmol) is added and the reaction mixture is stirred for an additional 2 hours (h) until the reaction is complete. The reaction is quenched by addition of a saturated solution of sodium bicarbonate (50 mL) and extracted three times with 150 mL of ethyl acetate. The organic layers are combined, washed with a saturated solution of sodium chloride (50 mL), dried over magnesium sulfate, filtered, and concentrated in vacuo. The residue is purified by a silica gel column chromatography (10-40% ethyl acetate/heptanes) to provide 4-[4-((E)-2-methoxycarbonyl-vinyl)-benzyl]-piperazine-1-carboxylic acid tert-butyl ester as a white solid (10.2 g).

### Example 2

### Preparation of 4-[4-((E)-2-hydroxycarbamoyl-vinyl)-benzyl]-piperazine-1-carboxylic acid tert-butyl ester (1)

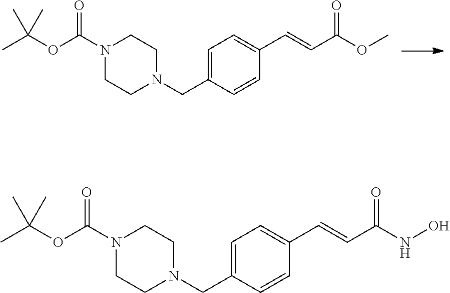

To a stirred solution of 4-[4-((E)-2-methoxycarbonyl-vinyl)-benzyl]-piperazine-1-carboxylic acid tert-butyl ester (4 mmol) in methanol (5 mL) at 0° C. is added sodium methoxide (5 equivalents, 20 mmol, 25% in methanol) and 10 equivalents of hydroxylamine (50% solution in water). The reaction is monitored by LCMS and, upon completion, is brought to pH 7-8 with 1 N hydrochloric acid. The solid is filtered, washed with water and dried in vacuo to yield 4-[4-((E)-2-hydroxycarbamoyl-vinyl)-benzyl]-piperazine-1-carboxylic acid tert-butyl ester. LCMS (m/z): 362.1 (M+1).

### Example 3

### General Procedure for the Synthesis of 2-Substituted indole-piperidine esters

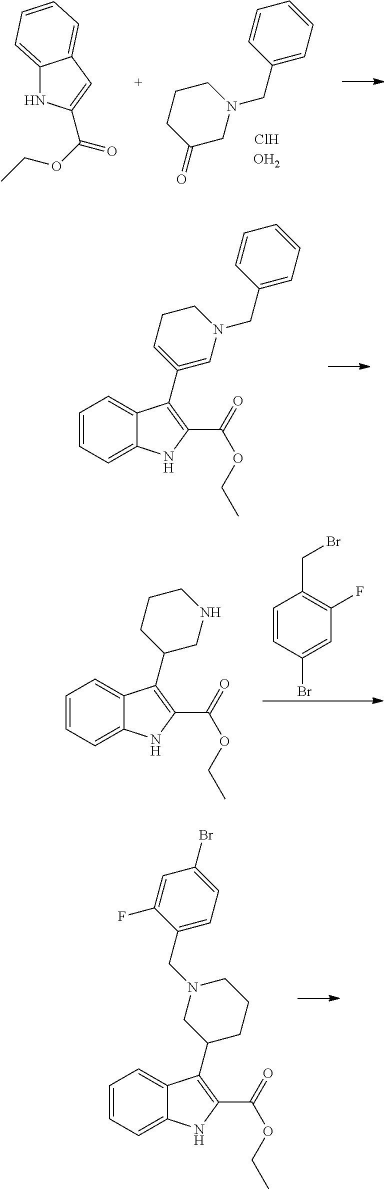

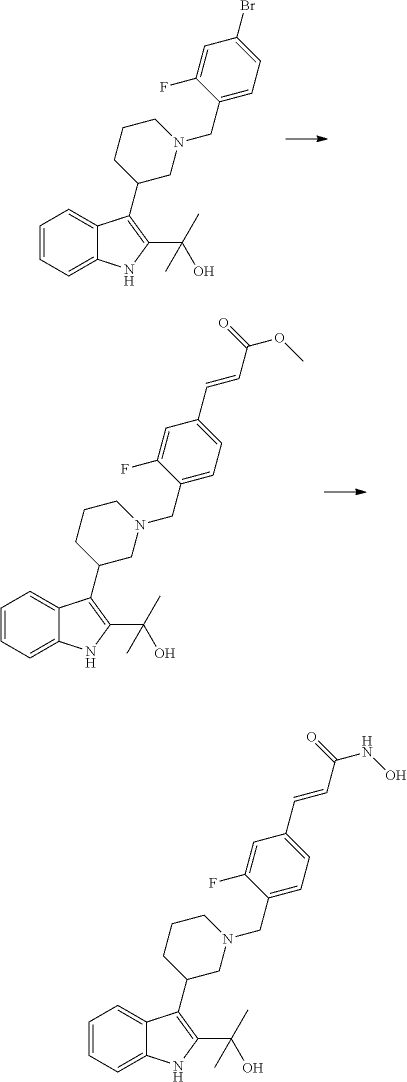

A solution of 1H-indole-2-carboxylic acid ethyl ester (5 g, 26.2 mmol), 1-benzyl-piperidin-3-one (12.9 g, 51.9 mmol) and concentrated sulfuric acid (1.45 mL, 26.2 mmol) in acetic acid (20 mL) is heated at 120° C. The reaction is monitored by thin layer chromatography. The mixture is diluted with ethyl acetate, washed with water and brine, dried over magnesium sulfate, filtered and concentrated in vacuo. The crude mixture is purified on a silica gel column chromatography to give a mixture of 3-(1-benzyl-1,2,5,6-tetrahydro-pyridin-3-yl)-1H-indole-2-carboxylic acid ethyl ester and 3-(1-benzyl-1,4,5,6-tetrahydro-pyridin-3-yl)-1H-indole-2-carboxylic acid ethyl ester (8.6 g, 91.2%). MS (m/z): 360.93 (M+1).

A mixture of 3-(1-benzyl-1,2,5,6-tetrahydro-pyridin-3-yl)-1H-indole-2-carboxylic acid ethyl ester, 3-(1-benzyl-1,4,5,6-tetrahydro-pyridin-3-yl)-1H-indole-2-carboxylic acid ethyl ester (8.6 g, 23.5 mmol) and Pd(OH)₂/C (6 g, 10% wt) in methanol (200 mL) is purged with nitrogen and filled with hydrogen at 60 PSI and the reaction mixture is agitated in a Parr shaker for 8 h. The mixture is filtered through a Celite pad, which is washed with methanol. The combined filtrates are concentrated and used for the next reaction without purification (5.9 g, 92.2%).

MS (m/z): 272.95 (M+1).

A solution of 3-piperidin-3-yl-1H-indole-2-carboxylic acid ethyl ester (2 g, 7.27 mmol), 4-bromo-1-bromomethyl-2-fluoro-benzene (2.92 g, 10.9 mmol) and triethylamine (3.04 mL, 21.8 mmol) in dichloroethane (20 mL) is stirred at room temperature for 4 h. The reaction mixture is diluted with ethyl acetate, washed with water and brine, dried over magnesium sulfate, filtered and concentrated. The crude mixture is purified by a silica gel column chromatography to give 3-[1-(4-bromo-2-fluoro-benzyl)-piperidin-3-yl]-1H-indole-2-carboxylic acid ethyl ester (3.5 g, 92.1%). MS (m/z): 460.72 (M+1).

A solution of 3-[1-(4-bromo-2-fluoro-benzyl)-piperidin-3-yl]-1H-indole-2-carboxylic acid ethyl ester (500 mg, 1.06 mmol) in tetrahydrofuran (10 mL) is treated with methylmagnesium bromide (3.2 mL of a 1 M solution, 3.2 mmol) and the resulting mixture is stirred at 0° C. for 2 h, treated with aqueous ammonium chloride slowly, and extracted with ethyl acetate several times. The combined organic layers are washed with brine, dried over magnesium sulfate, filtered, and concentrated, and the residue is purified by a silica gel column chromatography to give 2-{3-[1-(4-bromo-2-fluoro-benzyl)-piperidin-3-yl]-1H-indol-2-yl}-propan-2-ol (160 mg, 34.0%). MS (m/z): 446.73 (M+1).

A solution of 2-{3-[1-(4-bromo-2-fluoro-benzyl)-piperidin-3-yl]-1H-indol-2-yl}-propan-2-ol (160 mg, 0.356 mmol), methyl acrylate (62.8 mg, 0.715 mmol), Pd₂(dba)₃ (3.26 mg, 1%), P(t-Bu)₃-BF₄ (4.12 mg, 4%), Cy₂NMe (90 μL, 1.2 equivalents) and water (6.4 μL, 1.0 equivalent) in dioxane (3 mL) is heated at 100° C. by microwave for 30 min. The mixture is diluted with ethyl acetate, washed with water then brine, dried over magnesium sulfate, filtered and concentrated, and the residue is purified by a silica gel column chromatography to give (E)-3-(3-fluoro-4-{3-[2-(1-hydroxy-1-methyl-ethyl)-1H-indol-3-yl]-piperidin-1-ylmethyl}-phenyl)-acrylic acid methyl ester (156 mg, 97.4%). MS (m/z): 451.16 (M+1).

### Example 4

### Preparation of (E)-N-hydroxy-3-{4-[3-(1H-indol-3-yl)-piperidin-1-ylmethyl]-phenyl}-acrylamide (2)

Following procedures analogous to those described in Example 2, (E)-N-hydroxy-3-{4-[3-(1H-indol-3-yl)-piperidin-1-ylmethyl]-phenyl}-acrylamide is prepared from 1H-indole and 1-bromo-4-bromomethyl-benzene. LCMS (m/z): 376.00 (M+1).

### Example 5

### Preparation of (E)-N-hydroxy-3-{4-[(R)-3-(2-methyl-1H-indol-3-yl)-piperidin-1ylmethyl]-phenyl}-acrylamide (3)

Following procedures analogous to those described in Example 2, (E)-N-hydroxy-3-{4-[(R)-3-(2-methyl-1H-indol-3-yl)-piperidin-1ylmethyl]-phenyl}-acrylamide is prepared from 2-methyl-1H-indole and 1-bromo-4-bromomethyl-benzene. LCMS (m/z): 389.92 (M+1).

### Example 6

### Preparation of (E)-3-{3-fluoro-4-[3-(2-methyl-5-morpholin-4-yl-1H-indol-3-yl)-piperidin-1-ylmethyl]-phenyl}-N-hydroxy-acrylamide (4)

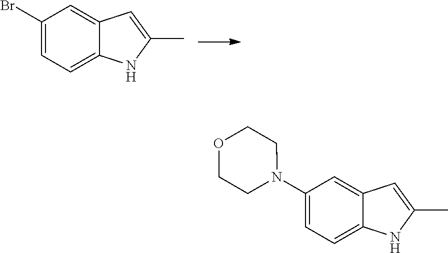

To a mixture of 5-bromo-2-methyl-1H-indole (4.40 g, 21 mmol) and morpholine (2.19 g, 25.1 mmol) in tetrahydrofuran (100 mL) is added a solution of lithium bis(trimethylsilyl)amide (1.0 M in tetrahydrofuran, 46.1 mmol) followed by (2′-dicyclohexylphosphanyl-biphenyl-2-yl)-dimethyl-amine (330 mg, 0.838 mmol). The resulting mixture is purged with nitrogen, treated with Pd₂(dba)₃ (192 mg, 0.210 mmol), and heated at 80° C. overnight. The reaction mixture is cooled to room temperature, concentrated and purified by a silica gel column chromatography to give 2-methyl-5-morpholin-4-yl-1H-indole (4.30 g, 95% yield). MS (m/z): 216.97 (M+1).

Following procedures analogous to those described in Example 2, (E)-3-{3-fluoro-4-[3-(2-methyl-5-morpholin-4-yl-1H-indol-3-yl)-piperidin-1-ylmethyl]-phenyl}-N-hydroxy-acrylamide is prepared from 2-methyl-5-morpholin-4-yl-1H-indole and 4-bromo-1-bromomethyl-2-fluoro-benzene. LCMS (m/z): 493.26 (M+1).

### Example 7

### Preparation of (E)-N-hydroxy-3-{4-[3-(2-phenyl-1H-indol-3-yl)-piperidin-1-ylmethyl]-phenyl}-acrylamide (5)

Following procedures analogous to those described in Example 2, (E)-N-hydroxy-3-{4-[3-(2-phenyl-1H-indol-3-yl)-piperidin-1-ylmethyl]-phenyl}-acrylamide is prepared from 2-phenyl-1H-indole and 1-bromo-4-bromomethyl-benzene. LCMS (m/z): 451.86 (M+1).

### Example 8

### Preparation of (E)-3-{4-[3-(2-tert-butyl-1H-indol-3-yl)-piperidin-1-ylmethyl]-phenyl}-N-hydroxy-acrylamide (6)

Following procedures analogous to those described in Example 2, (E)-3-{-4-[3-(2-tert-butyl-1H-indol-3-yl)-piperidin-1-ylmethyl]-phenyl}-N-hydroxy-acrylamide is prepared from 2-tent-butyl-1H-indole and 1-bromo-4-bromomethyl-benzene. LCMS (m/z): 431.94 (M+).

### Example 9

### Preparation of (E)-3-{4-[5-(2-tert-butyl-1H-indol-3-yl)-3,6-dihydro-2H-pyridin-1-ylmethyl]-phenyl}-N-hydroxy-acrylamide (7)

3-[1-(4-Bromo-benzyl)-1,2,5,6-tetrahydro-pyridin-3-yl]-2-tert-butyl-1H-indole is obtained during the synthesis of Example 8 and converted to (E)-3-{4-[5-(2-tert-butyl-1H-indol-3-yl)-3,6-dihydro-2H-pyridin-1-ylmethyl]-phenyl}-N-hydroxy-acrylamide by using procedures analogous to those described in Example 2. LCMS (m/z): 430.25 (M+1).

### Example 10

### Preparation of (E)-N-hydroxy-3-{4-[3-[(Z)-hydroxyimino]-5-(2-methyl-1H-indol-3-yl)-piperidin-1-ylmethyl]-phenyl}-acrylamide (8)

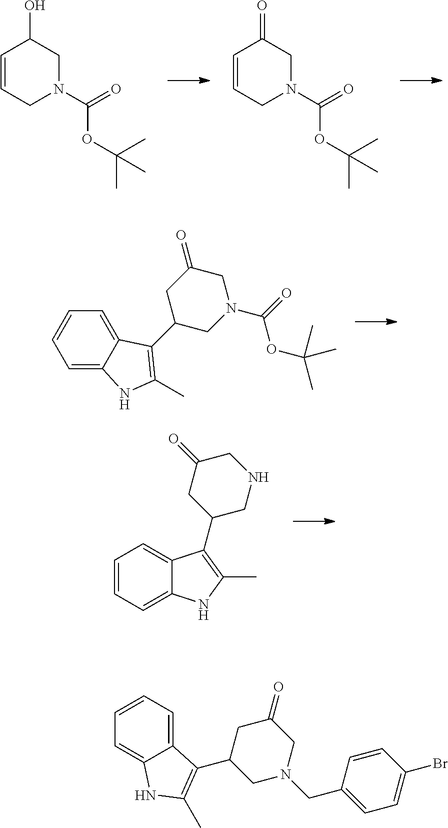

To a solution of N—BOC-3-hydroxyl-1,2,3,6-tetrahydropyridine (0.988 g, 4.96 mmol) in dichloromethane (20 mL) is added Dess-Martin reagent (2.49 g, 5.69 mmol) and the resulting mixture is stirred at room temperature. After 2 h, the reaction mixture is treated with 20% aqueous sodium thiosulfate solution (40 mL) and extracted with ethyl acetate (3×70 mL). The combined organics are washed with a saturated aqueous solution of sodium bicarbonate (2×50 mL), brine (50 mL), dried over magnesium sulfate, filtered, and concentrated, and the residue is purified via a silica gel column chromatography to give 3-oxo-3,6-dihydro-2H-pyridine-1-carboxylic acid tert-butyl ester (0.890 g, 91% yield). LCMS (m/z): 198.1 (M+1).

A solution of 3-oxo-3,6-dihydro-2H-pyridine-1-carboxylic acid tert-butyl ester (0.787 g, 3.99 mmol) and 2-methylindole (0.577 g, 4.31 mmol) in dichloromethane (5 mL) is treated with iodine portion-wise over 40 min (54 mg, 0.21 mmol). After 20 min, the reaction mixture is diluted with dichloromethane (380 mL), washed with a saturated aqueous solution of sodium thiosulfate (130 mL), dried over magnesium sulfate, filtered, and concentrated, and the residue is purified by a silica gel column chromatography to give 3-(2-methyl-1H-indol-3-yl)-5-oxo-piperidine-1-carboxylic acid tert-butyl ester (0.973 g, 74% yield). LCMS (m/z): 329.1 (M+1).

A solution of 3-(2-methyl-1H-indol-3-yl)-5-oxo-piperidine-1-carboxylic acid tert-butyl ester (0.973 g, 2.96 mmol) in dichloromethane (20 mL) is treated with trifluoroacetic acid (2 mL) and the resulting mixture is stirred at room temperature for 4 h. The reaction mixture is concentrated and used for the next reaction without purification.

To a solution of 5-(2-methyl-1H-indol-3-yl)-piperidin-3-one (677 mg, 2.97 mmol) in 1,2-dichloroethane (12 mL) is added triethylamine (3.0 mL, 21.6 mmol) followed by 4-bromobenzyl bromide (834 mg, 3.27 mmol) and the resulting mixture is stirred at room temperature for 2 h. The reaction mixture is diluted with dichloromethane (300 mL), washed with water (100 mL), brine (100 mL), dried over magnesium sulfate, filtered, and concentrated, and the residue is purified to give 1-(4-bromo-benzyl)-5-(2-methyl-1H-indol-3-yl)-piperidin-3-one (199 mg, 17% yield over two steps). LCMS (m/z): 398.9 (M+1).

Following procedures analogous to those described in Example 2, (E)-N-hydroxy-3-{4-[3-[(Z)-hydroxyimino]-5-(2-methyl-1H-indol-3-yl)-piperidin-1-ylmethyl]-phenyl}-acrylamide is prepared from 1-(4-bromo-benzyl)-5-(2-methyl-1H-indol-3-yl)-piperidin-3-one. LCMS (m/z): 419.0 (M+1).

### Example 11

### Preparation of (E)-N-hydroxy-3-{4-[3-(2-oxo-2,3-dihydro-1H-indol-3-yl)-piperidin-1-ylmethyl]-phenyl}-acrylamide (9)

Following procedures analogous to those described in Example 2, (E)-N-hydroxy-3-{4-[3-(2-oxo-2,3-dihydro-1H-indol-3-yl)-piperidin-1-ylmethyl]-phenyl}-acrylamide is prepared from 2-oxindole and 1-bromo-4-bromomethyl-benzene. LCMS (m/z): 392.96 (M+1).

### Example 12

### Preparation of (E)-3-{3-fluoro-4-[3-(1H-indol-3-yl)-piperidin-1-ylmethyl]-phenyl}-N-hydroxy-acrylamide (10)

Following procedures analogous to those described in Example 2, (E)-3-{3-fluoro-4-[3-(1H-indol-3-yl)-piperidin-1-ylmethyl]-phenyl}-N-hydroxy-acrylamide is prepared from 1H-indole and 4-bromo-1-bromomethyl-2-fluoro-benzene. LCMS (m/z): 393.83 (M+).

### Example 13

### Preparation of (E)-3-{4-[(S)-3-(2-tert-butyl-1H-indol-3-yl)-piperidin-1-ylmethyl]-3-fluoro-phenyl}-N-hydroxy-acrylamide (11)

Following procedures analogous to those described in Example 2, (E)-3-{4-[(S)-3-(2-tert-butyl-1H-indol-3-yl)-piperidin-1-ylmethyl]-3-fluoro-phenyl}-N-hydroxy-acrylamide is prepared from 2-tent-butyl-1H-indole and 4-bromo-1-bromomethyl-2-fluoro-benzene. LCMS (m/z): 450.25 (M+1).

### Example 14

### Preparation of (E)-3-{4-[3-(2-tert-butyl-1H-indol-3-yl)-piperidin-1-ylmethyl]-3-chloro-phenyl}-N-hydroxy-acrylamide (12)

Following procedures analogous to those described in Example 2, (E)-3-{4-[3-(2-tert-butyl-1H-indol-3-yl)-piperidin-1-ylmethyl]-3-chloro-phenyl}-N-hydroxy-acrylamide is prepared from 2-tent-butyl-1H-indole and 4-bromo-1-bromomethyl-2-chloro-benzene. LCMS (m/z): 466.22 (M+1).

### Example 15

### Preparation of (E)-3-(3-fluoro-4-{3-[2-(1-hydroxy-1-methyl-ethyl)-1H-indol-3-yl]-piperidin-1-ylmethyl}-phenyl)-N-hydroxy-acrylamide (13)

To a cooled (0° C.) solution of (E)-3-(3-fluoro-4-{3-[2-(1-hydroxy-1-methyl-ethyl)-1H-indol-3-yl]-piperidin-1-ylmethyl}-phenyl)-acrylic acid methyl ester (156 mg, 0.343 mmol) in methanol (3 mL) is added hydroxylamine 50% in water (0.226 mL, 3.42 mmol) and sodium methoxide 25% in methanol (0.370 mL, 1.71 mmol) and the mixture is stirred at 0° C. for 1.5 h. The reaction mixture is neutralized to pH 8 by 1 N HCl. The crude product is purified by a preparative HPLC to give (E)-3-(3-fluoro-4-{3-[2-(1-hydroxy-1-methyl-ethyl)-1H-indol-3-yl]-piperidin-1-ylmethyl}-phenyl)-N-hydroxy-acrylamide (68 mg, 43.9%). MS (m/z): 451.84 (M+1).

### Example 16

### Preparation of (E)-3-{3-fluoro-4-[3-(2-isopropenyl-1H-indol-3-yl)-piperidin-1-ylmethyl]-phenyl}-N-hydroxy-acrylamide (14)

(E)-3-{3-Fluoro-4-[3-(2-isopropenyl-1H-indol-3-yl)-piperidin-1-ylmethyl]-phenyl}-acrylic acid methyl ester is obtained during synthesis of Example 15 and converted to (E)-3-{3-fluoro-4-[3-(2-isopropenyl-1H-indol-3-yl)-piperidin-1-ylmethyl]-phenyl}-N-hydroxy-acrylamide by following procedures analogous to those described in Example 2. LCMS (m/z): 434.22 (M+1).

### Example 17

### Preparation of (E)-3-{3-fluoro-4-[3-(2-phenyl-1H-indol-3-yl)-piperidin-1-ylmethyl]-phenyl}-N-hydroxy-acrylamide (15)

Following procedures analogous to those described in Example 2, (E)-3-{3-fluoro-4-[3-(2-phenyl-1H-indol-3-yl)-piperidin-1-ylmethyl]-phenyl}-N-hydroxy-acrylamide is prepared from 2-phenyl-1H-indole and 4-bromo-1-bromomethyl-2-fluoro-benzene. LCMS (m/z): 470.23 (M+1).

### Example 18

### Preparation of N-hydroxy-3-{4-[3-(2-phenyl-1H-indol-3-yl)-piperidin-1-ylmethyl]-phenyl}-propionamide (16)

Following procedures analogous to those described in Example 2, N-hydroxy-3-{4-[3-(2-phenyl-1H-indol-3-yl)-piperidin-1-ylmethyl]-phenyl}-propionamide is prepared from 2-phenyl-1H-indole and 1-bromo-4-bromomethyl-benzene. LCMS (m/z): 454.25 (M+1)

### Example 19

### Preparation of 3-{1-[2-fluoro-4-((E)-2-hydroxycarbamoyl-vinyl)-benzyl]-piperidin-3-yl}-1H-indole-2-carboxylic acid ethyl ester (17)

3-[1-(4-Bromo-2-fluoro-benzyl)-piperidin-3-yl]-1H-indole-2-carboxylic acid ethyl ester is converted to the title compound following procedures analogous to those described in Example 2. LCMS (m/z): 465.85 (M+).

### Example 20

### Preparation of 3-{1-[2-fluoro-4-((E)-2-hydroxycarbamoyl-vinyl)-benzyl]-piperidin-3-yl}-1H-indole-2-carboxylic acid hydroxyamide (18)

3-[1-(4-Bromo-2-fluoro-benzyl)-piperidin-3-yl]-1H-indole-2-carboxylic acid ethyl ester is converted to 3-{1-[2-fluoro-4-((E)-2-hydroxycarbamoyl-vinyl)-benzyl]-piperidin-3-yl}-1H-indole-2-carboxylic acid hydroxyamide by following procedures analogous to those described in Example 2. LCMS (m/z): 452.93 (M+).

### Example 21

### Preparation of (E)-3-(3-fluoro-4-{3-[2-(morpholine-4-carbonyl)-1H-indol-3-yl]-piperidin-1-ylmethyl}-phenyl)-N-hydroxy-acrylamide (19)

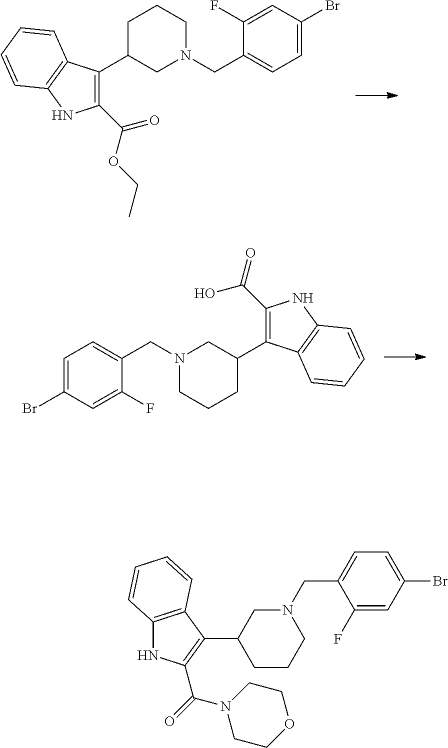

A solution of 3-[1-(4-bromo-2-fluoro-benzyl)-piperidin-3-yl]-1H-indole-2-carboxylic acid ethyl ester (600 mg, 1.29 mmol) in methanol (10 mL) is treated with an aqueous solution of sodium hydroxide (5 N, 3 mL) and the resulting mixture is stirred at room temperature overnight, diluted with ethyl acetate, and washed with an aqueous solution of hydrochloric acid (1 N) and brine, dried over magnesium sulfate, filtered, concentrated and used for the next reaction without purification.

To a solution of 3-[1-(4-bromo-2-fluoro-benzyl)-piperidin-3-yl]-1H-indole-2-carboxylic acid (200 mg, 0.459 mmol) in N,N-dimethylformamide (3 mL) are added O-benzotriazole-N,N,N′,N′-tetramethyl-uronium-hexafluoro-phosphate (261 mg, 0.689 mmol), N-hydroxybenzotriazole (93.0 mg, 0.689 mmol) and diisopropylethylamine (0.321 mL, 1.84 mmol). The resulting mixture is stirred for 20 min, treated with morpholine (49.5 mg, 0.551 mmol), and stirred at room temperature for 4 h. The reaction mixture is quenched with water, diluted with ethyl acetate, washed with water and brine, dried over magnesium sulfate, filtered and concentrated, and the residue is purified to give {3-[1-(4-bromo-2-fluoro-benzyl)-piperidin-3-yl]-1H-indol-2-yl}-morpholin-4-yl-methanone (210 mg, 91% yield). LCMS (m/z): 502.07 (M+1).

Following procedures analogous to those described in Example 2, (E)-3-(3-fluoro-4-{3-[2-(morpholine-4-carbonyl)-1H-indol-3-yl]-piperidin-1-ylmethyl}-phenyl)-N-hydroxy-acrylamide is prepared. LCMS (m/z): 507.14 (M+1).

### Example 22

### Preparation of (E)-N-hydroxy-3-{4-[3-(2-methyl-5-morpholin-4-yl-1H-indol-3-yl)-piperidin-1-ylmethyl]-phenyl}-acrylamide (20)

Following procedures analogous to those described in Example 2, (E)-N-hydroxy-3-{4-[3-(2-methyl-5-morpholin-4-yl-1H-indol-3-yl)-piperidin-1-ylmethyl]-phenyl}-acrylamide is prepared from 2-methyl-5-morpholin-4-yl-1H-indole (example 4) and 4-bromo-1-bromomethylbenzene. LCMS (m/z): 475.27 (M+1).

### Example 23

### Preparation of 3-{3-fluoro-4-[3-(2-isopropyl-1H-indol-3-yl)-piperidin-1-ylmethyl]-phenyl}-N-hydroxy-propionamide (21)

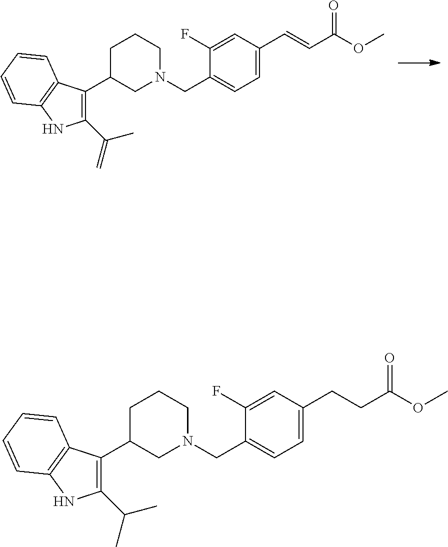

A mixture of (E)-3-{3-fluoro-4-[3-(2-isopropenyl-1H-indol-3-yl)-piperidin-1-ylmethyl]-phenyl}-acrylic acid methyl ester (530 mg, 1.21 mmol, obtained during the synthesis of Example 16) and palladium on carbon (300 mg, 10% wt) in methanol (10 mL) is purged with nitrogen and filled with hydrogen at 1 atmosphere. The reaction mixture is stirred for 8 h, and filtered through Celite, which is washed with methanol. The combined filtrate is concentrated and purified to give 3-{3-fluoro-4-[3-(2-isopropyl-1H-indol-3-yl)-piperidin-1-ylmethyl]-phenyl}-propionic acid methyl ester (200 mg, 38% yield). LCMS (m/z): 437.16 (M+1).

Following procedures analogous to those described in Example 2, 3-{3-fluoro-4-[3-(2-isopropyl-1H-indol-3-yl)-piperidin-1-ylmethyl]-phenyl}-N-hydroxy-propionamide is prepared. LCMS (m/z): 438.25 (M+1).

### Example 24

### Preparation of (E)-3-[4-(1,1-dimethyl-1,3,4,9-tetrahydro-beta-carbolin-2-ylmethyl)-phenyl]-N-hydroxy-acrylamide (22)

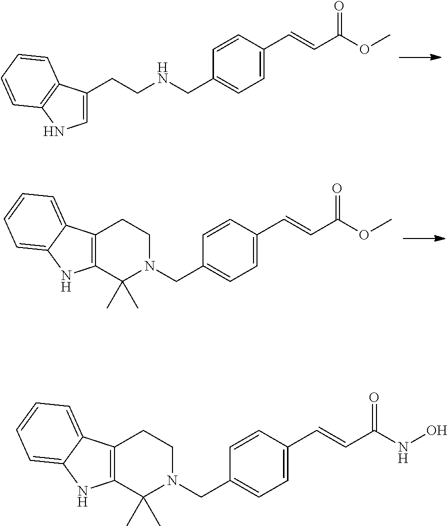

A mixture (E)-3-(4-{[2-(1H-indol-3-yl)-ethylamino]-methyl}-phenyl)-acrylic acid methyl ester (0.5 g, 1.45 mmol), acetone (543 mL, 7.40 mmol) and TiCl₄ (1 M in dichloromethane, 15 mL) in N,N-dimethylformamide (10 mL) is heated at 150° C. (microwave) for 30 min. The reaction mixture is diluted with ethyl acetate, washed with brine, dried over magnesium sulfate, filtered and concentrated in vacuo. The crude product is purified to give (E)-3-[4-(1,1-dimethyl-1,3,4,9-tetrahydro-beta-carbolin-2-ylmethyl)-phenyl]-acrylic acid methyl ester (200 mg, 36% yield). LCMS (m/z): 375.1 (M+1).

Following procedures analogous to those described in Example 2, (E)-3-[4-(1,1-dimethyl-1,3,4,9-tetrahydro-beta-carbolin-2-ylmethyl)-phenyl]-N-hydroxy-acrylamide is obtained. LCMS (m/z): 376.08 (M+1).

### Example 25

### Preparation of (E)-N-hydroxy-3-[4-(1-oxo-1,3,4,9-tetrahydro-beta-carbolin-2-ylmethyl)-phenyl]-acrylamide (23)

Following literature procedure (e.g., Synthesis, 1974, 354-356), intermediate 3-(2-amino-ethyl)-1H-indole-2-carboxylic acid ethyl ester hydrochloride salt is obtained. Following procedures analogous to those described in Examples 1 and 2, (E)-N-hydroxy-3-[4-(1-oxo-1,3,4,9-tetrahydro-beta-carbolin-2-ylmethyl)-phenyl]-acrylamide is obtained. MS (m/z): 362.1 (M+1).

### Example 26

### Preparation of (E)-N-hydroxy-3-{4-[2-(3-phenyl-isoxazol-5-yl)-morpholin-4-ylmethyl]-phenyl}-acrylamide (24)

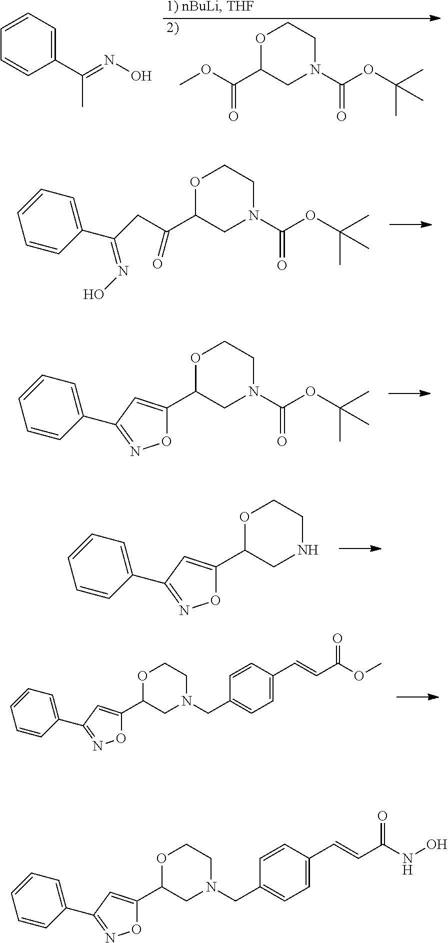

To a solution of acetophenone oxime (582 mg, 4.22 mmol) in tetrahydrofuran (5 mL) is added a solution of n-butyllithium (2.5 M in hexanes, 3.5 mL) at 0° C. and the resulting mixture is stirred at 0° C. for 3 h. To the reaction mixture is added a solution of morpholine-2,4-dicarboxylic acid 4-tent-butyl ester 2-methyl ester (0.5 g, 2.038 mmol) in tetrahydrofuran (5 mL). The resulting mixture is warmed to room temperature slowly and stirred overnight. The reaction mixture is treated with a saturated aqueous sodium bicarbonate (100 mL) and extracted with dichloromethane (3×100 mL). The combined organics are dried over magnesium sulfate, filtered and concentrated in vacuo, and the residue is purified via a silica gel column chromatography to give 2-{3-[(Z)-hydroxyimino]-3-phenyl-propionyl}-morpholine-4-carboxylic acid tert-butyl ester (670 mg, 94% yield). MS (m/z): 347.1 (M+1).

A solution of ketooxime 2-{3-[(Z)-hydroxyimino]-3-phenyl-propionyl}-morpholine-4-carboxylic acid tert-butyl ester (655 mg, 1.88 mmol) in dichloromethane (25 mL) is cooled to 0° C. and treated with triethylamine (0.80 mL, 5.75 mmol) and methanesulfonyl chloride (0.43 mL, 5.5 mmol). The resulting mixture is stirred at 0° C. for 6 h, treated with water (75 mL), and extracted with dichloromethane (3×100 mL). The combined organics are dried over magnesium sulfate, filtered and concentrated in vacuo. The crude product is used for the next reaction without purification. MS (m/z): 275.1 (M-tBu+1).

To a solution of 2-(3-phenyl-isoxazol-5-yl)-morpholine-4-carboxylic acid tert-butyl ester (300 mg, 0.908 mmol) in 1,4-dioxane (3 mL) is added a solution of HCl (1 mL of a 4 M solution in 1,4-dioxane) and the resulting mixture is stirred at room temperature for 2 h. The reaction mixture is concentrated and the HCl salt of 2-(3-phenyl-isoxazol-5-yl)-morpholine is precipitated from heptane/ethyl acetate (10:1) mixture (224 mg, 93% yield). MS (m/z): 231.1 (M+1).

Following procedures analogous to those described in Example 1, (E)-3-{-4-[2-(3-phenyl-isoxazol-5-yl)-morpholin-4-ylmethyl]-phenyl}-acrylic acid methyl ester is prepared. MS (m/z): 405.1 (M+1).

Following procedures analogous to those described in Example 2, (E)-N-hydroxy-3-{4-[2-(3-phenyl-isoxazol-5-yl)-morpholin-4-ylmethyl]-phenyl}-acrylamide is prepared. MS (m/z): 406.18 (M+1).

### Example 27

### Preparation of (E)-N-hydroxy-3-{4-[2-(3-phenyl-[1,2,4]oxadiazol-5-yl)-morpholin-4-ylmethyl]-phenyl}-acrylamide (25)

Starting from benzamidoxime and following procedures analogous to those described in Example 26, (E)-N-hydroxy-3-{4-[2-(3-phenyl-[1,2,4]oxadiazol-5-yl)-morpholin-4-ylmethyl]-phenyl}-acrylamide is prepared. MS (m/z): 407.17 (M+1)

### Example 28

### Preparation of (E)-3-[4-(4-benzyl-piperazin-1-ylmethyl)-phenyl]-N-hydroxy-acrylamide (26)

Starting from 1-benzyl-piperazine and following procedures analogous to those described in Example 26, (E)-3-[4-(4-benzyl-piperazin-1-ylmethyl)-phenyl]-N-hydroxy-acrylamide is prepared. LCMS (m/z): 352.14 (M+1).

### Example 29

### Preparation of (E)-3-{4-[4-(4-bromo-benzyl)-piperazin-1-ylmethyl]-phenyl}-N-hydroxy-acrylamide (27)

Starting from 1-(4-bromo-benzyl)-piperazine and following procedures analogous to those described in Example 26, (E)-3-{4-[4-(4-bromo-benzyl)-piperazin-1-ylmethyl]-phenyl}-N-hydroxy-acrylamide is prepared. LCMS (m/z): 428.1 (M−1).

### Example 30

### Preparation of 4-[4-((E)-2-hydroxycarbamoyl-vinyl)-benzyl]-piperazine-1-carboxylic acid ethyl ester (28)

Starting from piperazine-1-carboxylic acid ethyl ester and following procedures analogous to those described in Example 26, 4-[4-((E)-2-hydroxycarbamoyl-vinyl)-benzyl]-piperazine-1-carboxylic acid ethyl ester is prepared. LCMS (m/z): 334.1 (M+1).

### Example 31

### Preparation of 4-[4-((E)-2-hydroxycarbamoyl-vinyl)-benzyl]-piperazine-1-carboxylic acid benzyl ester (29)

Starting from piperazine-1-carboxylic acid benzyl ester and following procedures analogous to those described in Example 26, 4-[4-((E)-2-hydroxycarbamoyl-vinyl)-benzyl]-piperazine-1-carboxylic acid benzyl ester is prepared. LCMS (m/z): 396.16 (M+1).

### Example 32

### Preparation of (E)-N-hydroxy-3-{4-[4-(1-methyl-6-oxo-1,6-dihydro-pyridin-2-yl)-piperazin-1-ylmethyl]-phenyl}-acrylamide (30)

Following procedures analogous to those described in Example 1, (E)-3-{4-[4-(1-methyl-6-oxo-1,6-dihydro-pyridin-2-yl)-piperazin-1-ylmethyl]-phenyl}-acrylic acid methyl ester (0.117 g, 0.318 mmol) is prepared from 1-methyl-6-piperazin-1-yl-1H-pyridin-2-one (0.149 g, 0.771 mmol, 43% yield) as a yellowish white solid.

Following procedures analogous to those described in Example 2, (E)-N-hydroxy-3-{4-[4-(1-methyl-6-oxo-1,6-dihydro-pyridin-2-yl)-piperazin-1-ylmethyl]-phenyl}-acrylamide is prepared (54 mg, 0.147 mmol, 53% yield) as a white solid. LCMS (m/z): 369.0 (M+1).

### Example 33

### Preparation of (E)-3-{4-[4-(1-benzoyl-1H-benzoimidazol-2-yl)-piperazin-1-ylmethyl]-phenyl}-N-hydroxy-acrylamide (31)

Following procedures analogous to those described in Example 1, (E)-3-{4-[4-(1-benzoyl-1H-benzoimidazol-2-yl)-piperazin-1-ylmethyl]-phenyl}-acrylic acid methyl ester (0.255 g, 0.548 mmol, 75% yield) is prepared from phenyl-(2-piperazin-1-yl-benzoimidazol-1-yl)-methanone (0.236 g, 0.771 mmol) as a yellow solid.

Following procedures analogous to those described in Example 2, (E)-3-{4-[4-(1-benzoyl-1H-benzoimidazol-2-yl)-piperazin-1-ylmethyl]-phenyl}-N-hydroxy-acrylamide is prepared (6.2 mg, 0.013 mmol, 3% yield) as a yellow solid after preparative HPLC purification. LCMS (m/z): 482.1 (M+1).

### Example 34

### Preparation of (E)-3-{4-[4-(3-chloro-isoquinolin-1-yl)-piperazin-1-ylmethyl]-phenyl}-N-hydroxy-acrylamide (32)

Following procedures analogous to those described in Example 1, (E)-3-{4-[4-(3-chloro-isoquinolin-1-yl)-piperazin-1-ylmethyl]-phenyl}-acrylic acid methyl ester (0.224 g, 0.53 mmol, 72% yield) is prepared from 3-chloro-1-piperazin-1-yl-isoquinoline (0.2 g, 0.807 mmol) as a yellow solid.

Following procedures analogous to those described in Example 2, (E)-3-{4-[4-(3-chloro-isoquinolin-1-yl)-piperazin-1-ylmethyl]-phenyl}-N-hydroxy-acrylamide is prepared (1.2 mg, 0.003 mmol, 1% yield) as an off-white solid after preparative HPLC purification. LCMS (m/z): 423.0 (M+1).

### Example 35

### Preparation of (E)-N-hydroxy-3-(4-piperidin-1-ylmethyl-phenyl)-acrylamide (33)

Starting from piperidine and following procedures analogous to those described in Examples 1 and 2, (E)-N-hydroxy-3-(4-piperidin-1-ylmethyl-phenyl)-acrylamide is prepared. LCMS (m/z): 261.1 (M+1).

### Example 36

### Preparation of (E)-3-spiro[4H-3,1-benzoxazine-4,4′-piperidin]-2(1H)-1-ylmethyl)-phenyl]-N-hydroxy-acrylamide (34)

Following procedures analogous to those described in Example 1, (E)-3-spiro[4H-3,1-benzoxazine-4,4′-piperidin]-2(1H)-1-ylmethyl)-phenyl]-acrylic acid methyl ester (0.091 g, 0.232 mmol, 32% yield) is prepared from spiro[benzo[d][1,3]oxazine-4,4′-piperidin]-2(1H)-one (0.176 g, 0.807 mmol) as a white solid.

Following procedures analogous to those described in Example 2, (E)-3-spiro[4H-3,1-benzoxazine-4,4′-piperidin]-2(1H)-1-ylmethyl)-phenyl]-N-hydroxy-acryl amide is prepared (21 mg, 0.053 mmol, 26% yield) as a white solid. LCMS (m/z): 394.0 (M+1).

### Example 37

### Preparation of (E)-3-[4-(4-benzofuran-2-yl-piperidin-1-ylmethyl)-phenyl]-N-hydroxy-acrylamide (35)

Following procedures analogous to those described in Example 1, (E)-3-[4-(4-benzofuran-2-yl-piperidin-1-ylmethyl)-phenyl]-acrylic acid methyl ester (0.135 g, 0.360 mmol, 49% yield) is prepared from 4-benzofuran-2-yl-piperidine (0.155 g, 0.771 mmol) as a white solid.

Following procedures analogous to those described in Example 2, (E)-3-[4-(4-benzofuran-2-yl-piperidin-1-ylmethyl)-phenyl]-N-hydroxy-acrylamide is prepared (15 mg, 0.040 mmol, 11% yield) as a white solid. LCMS (m/z): 377.0 (M+1).

### Example 38

### Preparation of (E)-3-{4-[4-(7-bromo-5-methoxy-benzofuran-2-yl)-piperidin-1-ylmethyl]-phenyl}-N-hydroxy-acrylamide (36)

Following procedures analogous to those described in Example 1, (E)-3-{4-[4-(7-bromo-5-methoxy-benzofuran-2-yl)-piperidin-1-ylmethyl]-phenyl}-acrylic acid methyl ester (0.220 g, 0.454 mmol, 62% yield) is prepared from 4-(7-bromo-5-methoxy-benzofuran-2-yl)-piperidine (0.239 g, 0.771 mmol) as a white solid.

Following procedures analogous to those described in Example 2, (E)-3-{4-[4-(7-bromo-5-methoxy-benzofuran-2-yl)-piperidin-1-ylmethyl]-phenyl}-N-hydroxy-acrylamide is prepared (0.096 g, 0.198 mmol, 52% yield) as a white solid. LCMS (m/z): 486.0 (M+1).

### Example 39

### Preparation of (E)-3-[4-(3-cyclopropylmethyl-2,4-dioxo-1,3,8-triaza-spiro[4.5]dec-8-ylmethyl)-phenyl]-N-hydroxy-acrylamide (37)

Following procedures analogous to those described in Example 1, (E)-3-[4-(3-cyclopropylmethyl-2,4-dioxo-1,3,8-triaza-spiro[4.5]dec-8-ylmethyl)-phenyl]-acrylic acid methyl ester (0.102 g, 0.257 mmol, 35% yield) is prepared from 3-(cyclopropylmethyl)-1,3,8-triazaspiro[4.5]decane-2,4-dione (0.172 g, 0.771 mmol) as a white solid.

Following procedures analogous to those described in Example 2, (E)-3-[4-(3-cyclopropylmethyl-2,4-dioxo-1,3,8-triaza-spiro[4.5]dec-8-ylmethyl)-phenyl]-N-hydroxy-acrylamide is prepared (11 mg, 0.028 mmol, 13% yield) as a white solid. LCMS (m/z): 399.0 (M+1).

### Example 40

### Preparation of (E)-N-hydroxy-3-(4-(spiro[chroman-2,4′-piperidine]-1′-ylmethyl)phenyl)acrylamide (38)

Following procedures analogous to those described in Example 1, (E)-3-(4-(spiro[chroman-2,4′-piperidine]-1′-ylmethyl)phenyl)acrylic acid methyl ester (0.098 g, 0.260 mmol, 35% yield) is prepared from spiro[chroman-2,4′-piperidine] (0.157 g, 0.771 mmol) as a white solid.

Following procedures analogous to those described in Example 2, (E)-N-hydroxy-3-(4-(spiro[chroman-2,4′-piperidine]-1′-ylmethyl)phenyl)acrylamide is prepared (11 mg, 0.029 mmol, 11% yield) as a white solid. LCMS (m/z): 379.0 (M+1).

### Example 41

### Preparation of (E)-N-hydroxy-3-(4-((1-methylsulfonyl)spiro[indoline-3,4′-piperidine]-1′-yl)methyl)phenyl)acrylamide (39)

Following procedures analogous to those described in Example 1, (E)-3-(4-((1-methylsulfonyl)spiro[indoline-3,4′-piperidine]-1′-yl)methyl)phenyl)acrylic acid methyl ester (0.148 g, 0.336 mmol, 46% yield) is prepared from 1-(methylsulfonyl)spiro[indoline-3,4′-piperidine] (0.219 g, 0.771 mmol) as a white solid.

Following procedures analogous to those described in Example 2, the title compound is prepared (17 mg, 0.039 mmol, 13% yield) as a white solid. LCMS (m/z): 442.0 (M+1).

### Example 42

### Preparation of (E)-3-(4-{4-[4-chloro-2-(2-oxo-pyrrolidin-1-ylmethyl)-phenyl]-piperidin-1-ylmethyl}-phenyl)-N-hydroxy-acrylamide (40)

Following procedures analogous to those described in Example 1, (E)-3-(4-{4-[4-chloro-2-(2-oxo-pyrrolidin-1-ylmethyl)-phenyl]-piperidin-1-ylmethyl}-phenyl)-acrylic acid methyl ester (0.154 g, 0.330 mmol, 45% yield) is prepared from 1-(5-chloro-2-piperidin-4-ylbenzyl)-pyrrolidin-2-one (0.226 g, 0.771 mmol) as a white solid.

Following procedures analogous to those described in Example 2, (E)-3-(4-{4-[4-chloro-2-(2-oxo-pyrrolidin-1-ylmethyl)-phenyl]-piperidin-1-ylmethyl}-phenyl)-N-hydroxy-acrylamide is prepared (62 mg, 0.133 mmol, 46% yield) as a white solid. LCMS (m/z): 468.1 (M+1).

### Example 43

### Preparation of (E)-N-hydroxy-3-{4-[4-(1-oxo-3,4-dihydro-1H-isoquinolin-2-yl)-piperidin-1-ylmethyl]-phenyl}-acrylamide (41)

Following procedures analogous to those described in Example 1, (E)-3-{4-[4-(1-oxo-3,4-dihydro-1H-isoquinolin-2-yl)-piperidin-1-ylmethyl]-phenyl}-acrylic acid methyl ester (0.115 g, 0.284 mmol, 39% yield) is prepared from 2-piperidin-4-yl-3,4-dihydro-2H-isoquinolin-1-one (0.177 g, 0.771 mmol) as a white solid.

Following procedures analogous to those described in Example 2, (E)-N-hydroxy-3-{4-[4-(1-oxo-3,4-dihydro-1H-isoquinolin-2-yl)-piperidin-1-ylmethyl]-phenyl}-acrylamide is prepared (4 mg, 0.010 mmol, 4% yield) as a white solid after preparative HPLC purification. LCMS (m/z): 406.1 (M+1).

### Example 44

### Preparation of (E)-3-(4-{4-[(acetyl-methyl-amino)-methyl]-4-phenyl-piperidin-1-ylmethyl}-phenyl)-N-hydroxy-acrylamide (42)

Following procedures analogous to those described in Example 1, (E)-3-(4-{4-[(acetyl-methyl-amino)-methyl]-4-phenyl-piperidin-1-ylmethyl}-phenyl)-acrylic acid methyl ester (0.073 g, 0.174 mmol, 24% yield) is prepared from N-methyl-N-(4-phenyl-piperidin-4-ylmethyl)-acetamide (0.190 g, 0.771 mmol) as a colorless oil.

Following procedures analogous to those described in Example 2, (E)-3-(4-{4-[(acetyl-methyl-amino)-methyl]-4-phenyl-piperidin-1-ylmethyl}-phenyl)-N-hydroxy-acrylamide is prepared (19 mg, 0.045 mmol, 25% yield) as a yellow solid after preparative HPLC purification. LCMS (m/z): 422.1 (M+1).

### Example 45

### Preparation of (E)-N-hydroxy-3-(4-((1-methyl-2-oxospiro[indoline-3,4′-piperidine]-1′-yl)methyl)phenyl)acrylamide (43)

Following procedures analogous to those described in Example 1, (E)-3-(4-((1-methyl-2-oxospiro[indoline-3,4′-piperidine]-1′-yl)methyl)phenyl)acrylic acid methyl ester (0.218 g, 0.558 mmol, 76% yield) is prepared from 1-methylspiro[indoline-3,4′-piperidin]-2-one (0.167 g, 0.771 mmol) as a white solid.

Following procedures analogous to those described in Example 2, (E)-N-hydroxy-3-(4-((1-methyl-2-oxospiro[indoline-3,4′-piperidine]-1′-yl)methyl)phenyl)acrylamide is prepared (7 mg, 0.017 mmol, 4% yield) as a brown solid after preparative HPLC purification. LCMS (m/z): 392.0 (M+1).

### Example 46

### Preparation of (E)-N-hydroxy-3-{4-[4-(2-oxo-imidazolidin-1-yl)-piperidin-1-ylmethyl]-phenyl}-acrylamide (44)

Following procedures analogous to those described in Example 1, (E)-3-{4-[4-(2-oxo-imidazolidin-1-yl)-piperidin-1-ylmethyl]-phenyl}-acrylic acid methyl ester (0.119 g, 0.347 mmol, 47% yield) is prepared from 1-piperidin-4-yl-imidazolidin-2-one (0.130 g, 0.771 mmol) as a white solid.

Following procedures analogous to those described in Example 2, (E)-N-hydroxy-3-{4-[4-(2-oxo-imidazolidin-1-yl)-piperidin-1-ylmethyl]-phenyl}-acrylamide is prepared (3.5 mg, 0.010 mmol, 3% yield) as a white solid after preparative HPLC purification. LCMS (m/z): 345.0 (M+1).

### Example 47

### Preparation of (E)-3-{4-[4-(4-chloro-phenyl)-4-hydroxycarbamoylmethyl-piperidin-1-ylmethyl]-phenyl}-N-hydroxy-acrylamide (45)

Following procedures analogous to those described in Example 1, (E)-3-{4-[4-(4-chloro-phenyl)-4-hydroxycarbamoylmethyl-piperidin-1-ylmethyl]-phenyl}-acrylic acid methyl ester (0.138 g, 0.313 mmol, 61% yield) is prepared from [4-(4-chloro-phenyl)-piperidin-4-yl]-acetic acid methyl ester (0.136 g, 0.509 mmol) as a colorless oil.

Following procedures analogous to those described in Example 2, (E)-3-{4-[4-(4-chloro-phenyl)-4-hydroxycarbamoylmethyl-piperidin-1-ylmethyl]-phenyl}-N-hydroxy-acrylamide is prepared (8.3 mg, 0.018 mmol, 6% yield) as a white solid after preparative HPLC purification. LCMS (m/z): 444.0 (M+1).

### Example 48

### Preparation of (E)-N-hydroxy-3-{4-[4-(10-oxo-9,10-dihydro-1-thia-benzo[f]azulen-4-ylidene)-piperidin-1-ylmethyl]-phenyl}-acrylamide (46)

Following procedures analogous to those described in Example 1, (E)-3-{4-[4-(10-oxo-9,10-dihydro-1-thia-benzo[f]azulen-4-ylidene)-piperidin-1-ylmethyl]-phenyl}-acrylic acid methyl ester (0.197 g, 0.419 mmol, 57% yield) is prepared from 4-piperidin-4-ylidene-4,9-dihydro-1-thia-benzo[f]azulen-10-one (0.228 g, 0.771 mmol) as a yellow solid.

Following procedures analogous to those described in Example 2, (E)-N-hydroxy-3-{4-[4-(10-oxo-9,10-dihydro-1-thia-benzo[f]azulen-4-ylidene)-piperidin-1-ylmethyl]-phenyl}-acrylamide is prepared (2.2 mg, 0.005 mmol, 1% yield) as an off-white solid after preparative HPLC purification. LCMS (m/z): 471.2 (M+1).

### Example 49

### Preparation of (E)-N-hydroxy-3-(4-morpholin-4-ylmethyl-phenyl)-acrylamide (47)

Starting from morpholine and following procedures analogous to those described in Examples 1 and 2, (E)-N-hydroxy-3-(4-morpholin-4-ylmethyl-phenyl)-acrylamide is prepared. LCMS (m/z): 263.0 (M+1).

### Example 50

### Preparation of (E)-3-[4-(1,4-dioxa-8-aza-spiro[4.5]dec-8-ylmethyl)-phenyl]-N-hydroxy-acrylamide (48)

Starting from 1,4-dioxa-8-aza-spiro[4.5]decane and following procedures analogous to those described in Examples 1 and 2, (E)-3-[4-(1,4-dioxa-8-aza-spiro[4.5]dec-8-ylmethyl)-phenyl]-N-hydroxy-acrylamide is prepared. LCMS (m/z): 319.1 (M+1).

### Example 51

### Preparation of (E)-3-[4-(4-benzyl-piperidin-1-ylmethyl)-phenyl]-N-hydroxy-acrylamide (49)

Starting from 4-benzyl-piperidine and following procedures analogous to those described in Examples 1 and 2, (E)-3-[4-(4-benzyl-piperidin-1-ylmethyl)-phenyl]-N-hydroxy-acrylamide is prepared. LCMS (m/z): 351.16 (M+1).

### Example 52

### Typical Procedure for the Formation of Amide, Sulfonamide and Urea Derivatives of (E)-3-(4-piperazin-1-ylmethyl-phenyl)-acrylic acid methyl ester

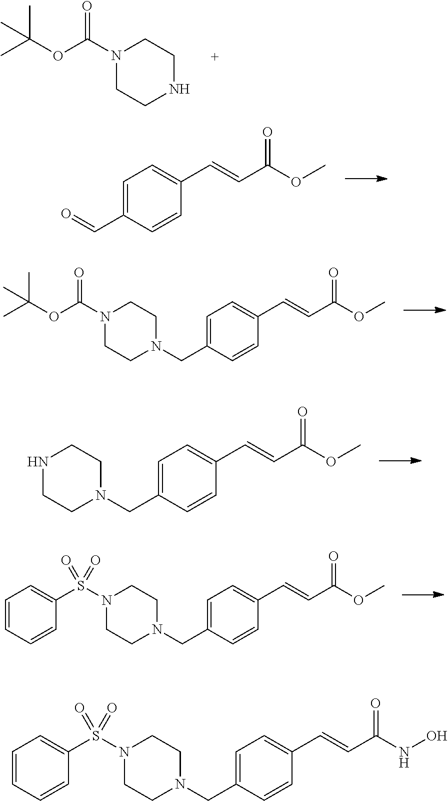

### Step A: preparation of (E)-3-(4-piperazin-1-ylmethyl-phenyl)-acrylic acid methyl ester

To a stirred solution of 4-[4-((E)-2-methoxycarbonyl-vinyl)-benzyl]-piperazine-1-carboxylic acid tent-butyl ester (11.17 g, 29.4 mmol) in dichloromethane (100 mL) is added 4 N HCl in dioxane (200 mL). The reaction mixture is stirred for 4 h, concentrated in vacuo, triturated with ether, and filtered to yield 10.3 g of a white solid. The solid is suspended in 500 mL dichloromethane and washed with saturated sodium bicarbonate (2×250 mL), dried with magnesium sulfate, filtered and concentrated in vacuo to provide (E)-3-(4-piperazin-1-ylmethyl-phenyl)-acrylic acid methyl ester as a white solid (6.52 g, 80% yield).

### Step B: Preparation of Amide, Sulfonamide and Urea Derivatives of (E)-3-(4-piperazin-1-ylmethyl-phenyl)-acrylic acid methyl ester

To a solution of (E)-3-(4-piperazin-1-ylmethyl-phenyl)-acrylic acid methyl ester (250 mg, 0.96 mmol) in dichloromethane is added SiliaBond Dimethylamine (961 mg, 1.4 mmol) followed by 1 mmol of the appropriate acid chloride, sulfonyl chloride or isocyanate. The mixture is stirred for 2 h, rinsed and concentrated in vacuo. Purification via a silica gel column chromatography (ethyl acetate/heptanes) provides the desired products as analytically pure material.

Following the general procedures above, the following compounds are prepared.

| Compound | Name | MS (M + 1) |
|---|---|---|
| 50 | (E)-3-[4-(4-Benzenesulfonyl-piperazin-1-ylmethyl)- | 402.1 |
| | phenyl]-N-hydroxy-acrylamide | |
| 51 | (E)-3-[4-(4-Benzoyl-piperazin-1-ylmethyl)-phenyl]-N- | 366.2 |
| | hydroxy-acrylamide | |
| 52 | (E)-N-Hydroxy-3-[4-(4-propionyl-piperazin-1-ylmethyl)- | 318.12 |
| | phenyl]-acrylamide | |
| 53 | (E)-3-{4-[4-(3,3-Dimethyl-butyryl)-piperazin-1-ylmethyl]- | 360.2 |
| | phenyl}-N-hydroxy-acrylamide | |
| 54 | (E)-3-[4-(4-Cyclohexanecarbonyl-piperazin-1-ylmethyl)- | 372.17 |
| | phenyl]-N-hydroxy-acrylamide | |
| 55 | (E)-N-Hydroxy-3-{4-[4-(toluene-4-sulfonyl)-piperazin-1- | 416.1 |
| | ylmethyl]-phenyl}-acrylamide | |
| 56 | (E)-N-Hydroxy-3-{4-[4-(toluene-3-sulfonyl)-piperazin-1- | 416.04 |
| | ylmethyl]-phenyl}-acrylamide | |
| 57 | (E)-3-{4-[4-(4-Fluoro-benzenesulfonyl)-piperazin-1- | 420.06 |
| | ylmethyl]-phenyl}-N-hydroxy-acrylamide | |
| 58 | (E)-3-[4-(4-Benzenesulfonyl-3,4-dihydro-2H-quinoxalin-1- | 450.2 |
| | ylmethyl)-phenyl]-N-hydroxy-acrylamide | |
| 59 | 4-[4-((E)-2-Hydroxycarbamoyl-vinyl)-benzyl]-3,4-dihydro- | 429.2 |
| | 2H-quinoxaline-1-carboxylic acid phenylamide | |
| 60 | (E)-3-[4-(4-Cyclohexanecarbonyl-3,4-dihydro-2H- | 420.0 |
| | quinoxalin-1-ylmethyl)-phenyl]-N-hydroxy-acrylamide | |
| 61 | 4-[4-((E)-2-Hydroxycarbamoyl-vinyl)-benzyl]-3,4-dihydro- | 443.2 |
| | 2H-quinoxaline-1-carboxylic acid benzylamide | |

### Example 55

### HDAC Inhibition Assay

The baculovirus donor vector pFB-GSTX3 is used to generate a recombinant baculovirus that can express the HDAC polypeptide. Transfer vectors containing the HDAC coding region are transfected into the DH10Bac cell line (GIBCO) and plated on selective agar plates. Colonies without insertion of the fusion sequence into the viral genome (carried by the bacteria) are blue. Single, white colonies are picked and viral DNAs (bacmid) are isolated from the bacteria by standard plasmid purification procedures. Sf9 cells or Sf21 (American Type Culture Collection) cells are then transfected in 25 cm³ flasks with the viral DNA using Cellfectin reagent.

### Determination of Small Scale Protein Expression in Sf9 Cells

Virus-containing media is collected from the transfected cell culture and used for infection to increase its titer. Virus-containing media obtained after two rounds of infection is used for large-scale protein expression. For large-scale protein expression 100 cm² round tissue culture plates are seeded with 5×10⁷ cells/plate and infected with 1 mL of virus-containing media (at an approximately MOI of 5). After 3 days, the cells are scraped off the plate and centrifuged at 500 rpm for 5 minutes. Cell pellets from 10-20, 100 cm² plates, are re-suspended in 50 mL of ice-cold lysis buffer (25 mM tris-HCl, pH 7.5, 2 mM EDTA, 1% NP-40, 1 mM DTT, 1 mM P MSF). The cells are stirred on ice for 15 minutes and then centrifuged at 5,000 rpms for 20 minutes.

### Purification of GST-Tagged Proteins

The centrifuged cell lysate is loaded onto a 2 mL glutathione-sepharose column (Pharmacia) and is washed thrice with 10 mL of 25 mM tris-HCl, pH 7.5, 2 mM EDTA, 1 mM DTT, 200 mM NaCl. The GST-tagged proteins are then eluted by 10 applications (1 mL each) of 25 mM tris-HCl, pH 7.5, 10 mM reduced-glutathione, 100 mM NaCl, 1 mM DTT, 10% glycerol and stored at −70° C.

### Enzyme Activity Measurement

HDAC assays with purified GST-HDAC protein are carried out in a final volume of 30 μL containing 15 ng of GST-HDAC protein, 20 mM tris-HCl, pH 7.5, 1 mM MnCl₂, 10 mM MgCl₂, 1 mM DTT, 3 μg/mL poly(Glu,Tyr) 4:1, 1% DMSO, 2.0 μM ATP (γ-[³³P]-ATP 0.1 μCi). The activity is assayed in the presence or absence of inhibitors. The assay is carried out in 96-well plates at ambient temperature for 15 minutes under conditions described below and terminated by the addition of 20 μL of 125 mM EDTA. Subsequently, 40 μL of the reaction mixture are transferred onto EMMOBILON-PVDF membrane (Millipore) previously soaked for 5 minutes with methanol, rinsed with water, then soaked for 5 minutes with 0.5% H₃PO₄ and mounted on vacuum manifold with disconnected vacuum source. After spotting all samples, a vacuum is connected and each well rinsed with 200 μL 0.5% H₃PO₄. Membranes are removed and washed four times on a shaker with 1.0% H₃PO₄, once with ethanol. Membranes are counted after drying at ambient temperature, mounting in Packard TopCount 96-well frame, and addition of 10 μL/well of MICROSCINT™ (Packard). IC50 values are calculated by linear regression analysis of the percentage inhibition of each compound in duplicate, at 4 concentrations (usually 0.01, 0.1, 1 and 10 μM).

### IC₅₀ Calculations

Input: 3×4 μL stopped assay on IMMOBILON membrane, not washed
Background (3 wells): assay with H₂O instead of enzyme
Positive control (4 wells): 3% DMSO instead of compound
Bath control (1 well): no reaction mix

IC50 values are calculated by logarithmic regression analysis of the percentage inhibition of each compound at 4 concentrations (usually 3- or 10-fold dilution series starting at 10 μM). In each experiment, the actual inhibition by reference compound is used for normalization of IC50 values to the basis of an average value of the reference inhibitor:

Normalized IC50=measured IC50 average ref. IC50/measured ref. IC50

Example: Reference inhibitor in experiment 0.4 μM, average 0.3 μM,

Test compound in experiment 1.0 μL, normalization: 0.3/0.4=0.75 μM

For example, known HDAC inhibitors or a synthetic derivative thereof may be used as reference compounds.

Using this protocol, the compounds of the present teachings are found to show IC50 values for HDAC inhibition in the range from about 0.0008 μL to about 100 μL, or about 0.0008 μL to about 50 μL, including, for example, the range from about 0.0008 μL to about 2 μL or less.

Table 2 provides assay results of exemplified compounds.

TABLE 2

| | HDAC-1 | HCT116 |
|---|---|---|
| Compound | IC₅₀ (nM) | IC₅₀ (nM) |
| 1 | | |
| 2 | 9 | 32 |
| 3 | 6 | 12 |
| 4 | 11 | 13 |
| 5 | 6 | 2.1 |
| 6 | 3 | 0.3 |
| 7 | 9 | 12 |
| 8 | 17 | 300 |
| 9 | 8 | 48 |
| 10 | 140 | 330 |
| 11 | 7.5 | 0.8 |
| 12 | 19 | 5 |
| 13 | 6 | 2.8 |
| 14 | 7 | 6 |
| 15 | 11 | 5.2 |
| 16 | 2.2 | |
| 17 | 44 | 14 |
| 18 | 31 | 37 |
| 19 | 22 | 500 |
| 20 | 7 | 13 |
| 21 | 6 | 21 |
| 22 | 150 | |
| 23 | | |
| 24 | 32 | 470 |
| 25 | 39 | |
| 26 | 100 | |
| 27 | 130 | |
| 28 | 170 | |
| 29 | 140 | |
| 30 | 170 | |
| 31 | 231 | |
| 32 | | |
| 33 | 100 | |
| 34 | 32 | |
| 35 | 46 | |
| 36 | 500 | |
| 37 | <430 | |
| 38 | 110 | |
| 39 | 27 | |
| 40 | 27 | |
| 41 | 16 | |
| 42 | 70 | |
| 43 | 24 | |
| 44 | 50 | |
| 45 | 110 | |
| 46 | | |
| 47 | 300 | |
| 48 | 130 | |
| 49 | 37 | |
| 50 | 230 | |
| 51 | 110 | |
| 52 | 350 | |
| 53 | 160 | |
| 54 | 130 | |
| 55 | 230 | |
| 56 | 260 | |
| 57 | 260 | |
| 58 | 290 | |
| 59 | 320 | |
| 60 | 220 | |
| 61 | 250 | |

As those skilled in the art will appreciate, numerous changes and modifications can be made to the above-described embodiments of the present teachings without departing from the spirit of the present teachings. It is intended that all such variations fall within the scope of the present teachings.

1. A compound of Formula I: 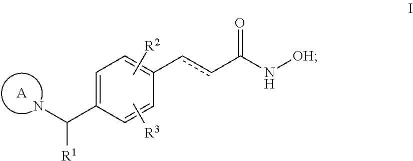
or a pharmaceutically acceptable salt, hydrate, or ester thereof,
wherein: [symbol]
is a) a single bond or b) a double bond;
ring A, including the nitrogen atom (N), is a 6-14 membered cycloheteroalkyl group optionally substituted with 1-5 R⁴ groups;
R¹ is a) H, b) a C₁₋₁₀ alkyl group, c) a C₂₋₁₀ alkenyl group, d) a C₂₋₁₀ alkynyl group, e) a C₃₋₁₄ cycloalkyl group, or f) a 3-14 membered cycloheteroalkyl group, wherein each of b)-f) optionally is substituted with 1-4 -L-R⁸ groups;
R² and R³ independently are a) H or b) halogen;
R⁴, at each occurrence, is a) halogen, b) oxo, c) ═NR⁶, d) ═CR⁶R⁷, e) —OR⁶, f) —NR⁶R⁷, g) —C(O)R⁵, h) —C(O)OR⁶, i) —C(O)NR⁶R⁷, j) —S(O)ₘR⁵, k) a C₁₋₁₀ alkyl group, l) a C₂₋₁₀ alkenyl group, m) a C₂₋₁₀ alkynyl group, n) a C₃₋₁₄ cycloalkyl group, o) a C₆₋₁₄ aryl group, p) a 3-14 membered cycloheteroalkyl group, or q) a 5-14 membered heteroaryl group, wherein each of k)-q) optionally is substituted with 1-4 groups independently selected from R⁸ and -L-R⁸, or
two R⁴ groups, taken together with the atom to which each R⁴ group is attached and any intervening ring atoms, form a) a C₃₋₁₄ cycloalkyl group, b) a C₆₋₁₄ aryl group, c) a 3-14 membered cycloheteroalkyl group, or d) a 5-14 membered heteroaryl group, wherein each of a)-d) optionally is substituted with 1-4 groups independently selected from R⁸ and -L-R⁸;
R⁵, at each occurrence, is a) H, b) a C₁₋₁₀ alkyl group, c) a C₂₋₁₀ alkenyl group, d) a C₂₋₁₀ alkynyl group, f) a C₃₋₁₄ cycloalkyl group, g) a C₆₋₁₄ aryl group, h) a 3-14 membered cycloheteroalkyl group, or i) a 5-14 membered heteroaryl group, wherein each of b)-i) optionally is substituted with 1-4 groups independently selected from R⁸ and -L-R⁸;
R⁶ and R⁷, at each occurrence, independently are a) H, b) —OR⁹, c) —NR⁹R¹⁰, d) —C(O)R⁹, e) —C(O)OR⁹, f) —C(O)NR⁹R¹⁰, g) —S(O)ₘR⁹, h) a C₁₋₁₀ alkyl group, i) a C₂₋₁₀ alkenyl group, j) a C₂₋₁₀ alkynyl group, k) a C₃₋₁₄ cycloalkyl group, 1) a C₆₋₁₄ aryl group, m) a 3-14 membered cycloheteroalkyl group, or n) a 5-14 membered heteroaryl group, wherein each of h)-n) optionally is substituted with 1-4 groups independently selected from R⁸ and -L-R⁸, or
R⁶ and R⁷, taken together with the atom to which each group is attached, form a) a C₃₋₁₄ cycloalkyl group, b) a C₆₋₁₄ aryl group, c) a 3-14 membered cycloheteroalkyl group, or d) a 5-14 membered heteroaryl group, each of which optionally is substituted with 1-4 groups independently selected from R⁸ and -L-R⁸;
R⁸, at each occurrence, is a) halogen, b) —CN, c) —NO₂, d) oxo, e) ═N-L-R⁹, f) —O-L-R⁹, g) —NR¹⁰-L-R⁹, h) —C(O)R⁹, i) —C(O)O-L-R⁹, j) —C(O)NR¹⁰-L-R⁹, k) —S(O)ₘR⁹, l) —Si(C₁₋₁₀ alkyl)₃, m) a C₁₋₁₀ alkyl group, n) a C₂₋₁₀ alkenyl group, o) a C₂₋₁₀ alkynyl group, p) a C₃₋₁₄ cycloalkyl group, q) a C₆₋₁₄ aryl group, r) a 3-14 membered cycloheteroalkyl group, or s) a 5-14 membered heteroaryl group, wherein each of the C₁₋₁₀ alkyl groups, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4 groups independently selected from R¹³ and -L-R¹³;
R⁹ and R¹⁰, at each occurrence, independently are a) H, b) —OR¹¹, c) —NR¹¹R¹², d) —C(O)R¹¹, e) —C(O)OR¹¹, f) —C(O)NR¹¹R¹², g) —S(O)ₘR¹¹, h) a C₁₋₁₀ alkyl group, i) a C₂₋₁₀ alkenyl group, j) a C₂₋₁₀ alkynyl group, k) a C₃₋₁₄ cycloalkyl group, l) a C₆₋₁₄ aryl group, m) a 3-14 membered cycloheteroalkyl group, or n) a 5-14 membered heteroaryl group, wherein each of h)-n) optionally is substituted with 1-4 groups independently selected from R¹³ and -L-R¹³;
R¹¹ and R¹², at each occurrence, independently are a) H, b) a C₁₋₁₀ alkyl group, c) a C₂₋₁₀ alkenyl group, d) a C₂₋₁₀ alkynyl group, e) a C₃₋₁₄ cycloalkyl group, f) a C₆₋₁₄ aryl group, g) a 3-14 membered cycloheteroalkyl group, or h) a 5-14 membered heteroaryl group, wherein each of b)-h) optionally is substituted with 1-4 groups independently selected from R¹³ and -L-R¹³;
R¹³, at each occurrence, is a) halogen, b) —CN, c) —NO₂, d) oxo, e) —OH, f) —NH₂, g) —NH(C₁₋₁₀ alkyl), h) —N(C₁₋₁₀ alkyl)₂, i) —CHO, j) —C(O)—C₁₋₁₀ alkyl, k) —C(O)OH, l) —C(O)—OC₁₋₁₀ alkyl, m) —C(O)SH, n) —C(O)—SC₁₋₁₀ alkyl, o) —C(O)NH₂, p) —C(O)NH(C₁₋₁₀ alkyl), q) —C(O)N(C₁₋₁₀ alkyl)₂, r) —C(S)H, s) —C(S)—C₁₋₁₀ alkyl, t) —C(S)NH₂, u) —C(S)NH(C₁₋₁₀ alkyl), v) —C(S)N(C₁₋₁₀ alkyl)₂, w) —C(NH)H, x) —C(NH)C₁₋₁₀ alkyl, y) —C(NH)NH₂, z) —C(NH)NH(C₁₋₁₀ alkyl), aa) —C(NH)N(C₁₋₁₀ alkyl)₂, ab) —C(NC₁₋₁₀ alkyl)H, ac) —C(NC₁₋₁₀ alkyl)-C₁₋₁₀ alkyl, ad) —C(NC₁₋₁₀ alkyl)NH(C₁₋₁₀ alkyl), ae) —C(NC₁₋₁₀ alkyl)N(C₁₋₁₀ alkyl)₂, af) —S(O)ₘH, ag) —S(O)ₘ—C₁₋₁₀ alkyl, ah) —S(O)₂OH, ai) —S(O)ₘ—OC₁₋₁₀ alkyl, aj) —S(O)ₘNH₂, ak) —S(O)ₘNH(C₁₋₁₀ alkyl), al) —S(O)ₘN(C₁₋₁₀ alkyl)₂, am) —Si(C₁₋₁₀ alkyl)₃, an) a C₁₋₁₀ alkyl group, ao) a C₂₋₁₀ alkenyl group, ap) a C₂₋₁₀ alkynyl group, aq) a C₁₋₁₀ alkoxy group, ar) a C₁₋₁₀ haloalkyl group, as) a C₃₋₁₄ cycloalkyl group, at) a C₆₋₁₄ aryl group, au) a 3-14 membered cycloheteroalkyl group, or av) a 5-14 membered heteroaryl group;
L, at each occurrence, is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, e) a divalent C₁₋₁₀ alkoxy group, or f) a covalent bond; and m, at each occurrence, is 0, 1, or 2.

2. The compound of claim 1, or a pharmaceutically acceptable salt, hydrate, or ester thereof, the compound having Formula Ia or Formula Ib: 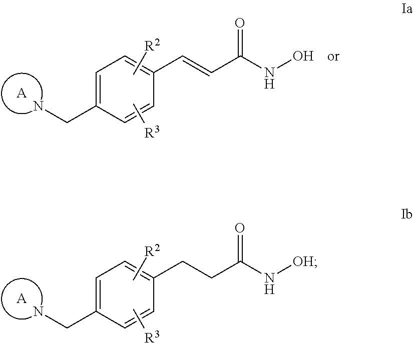
wherein ring A, R², and R³ are as defined in claim 1.

3. The compound of claim 1, or a pharmaceutically acceptable salt, hydrate, or ester thereof, wherein ring A is selected from: 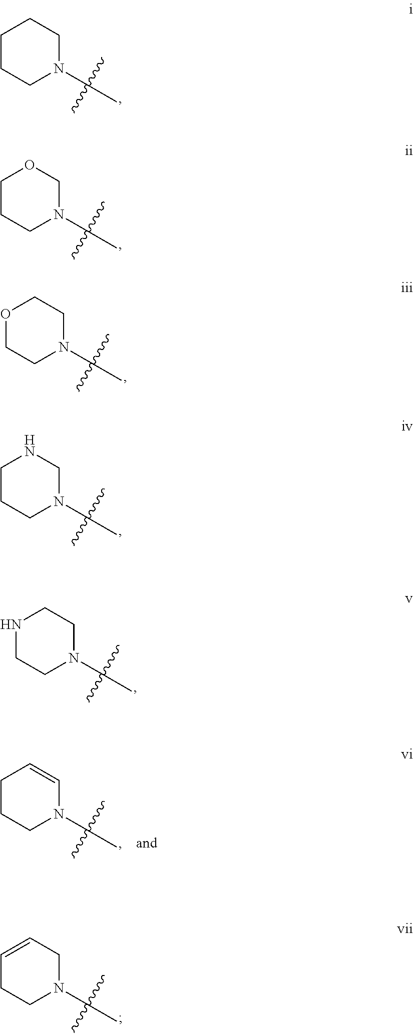
wherein each of i-vii optionally is substituted with 1-5 R⁴ groups, wherein R⁴ is as defined in claim 1.

4. The compound of claim 1, or a pharmaceutically acceptable salt, hydrate, or ester thereof, wherein ring A is selected from i, iii, v, and vii, each of which optionally is substituted with 1-5 R⁴ groups, wherein R⁴ is as defined in claim 1.

5. The compound of claim 1, or a pharmaceutically acceptable salt, hydrate, or ester thereof, wherein two R⁴ groups, taken together with the atom to which each R⁴ group is attached and any intervening ring atoms, form a C₆₋₁₄ aryl group, a 3-14 membered cycloheteroalkyl group, or a 5-14 membered heteroaryl group, wherein each of the C₆₋₁₄ aryl group, the 3-14 membered cycloheteroalkyl group, and the 5-14 membered heteroaryl group optionally is substituted with 1-4 groups independently selected from R⁸ and -L-R⁸, wherein L and R⁸ are as defined in claim 1.

6. The compound of claim 5, or a pharmaceutically acceptable salt, hydrate, or ester thereof, wherein the C₆₋₁₄ aryl group, the 3-14 membered cycloheteroalkyl group, and the 5-14 membered heteroaryl group are selected from: 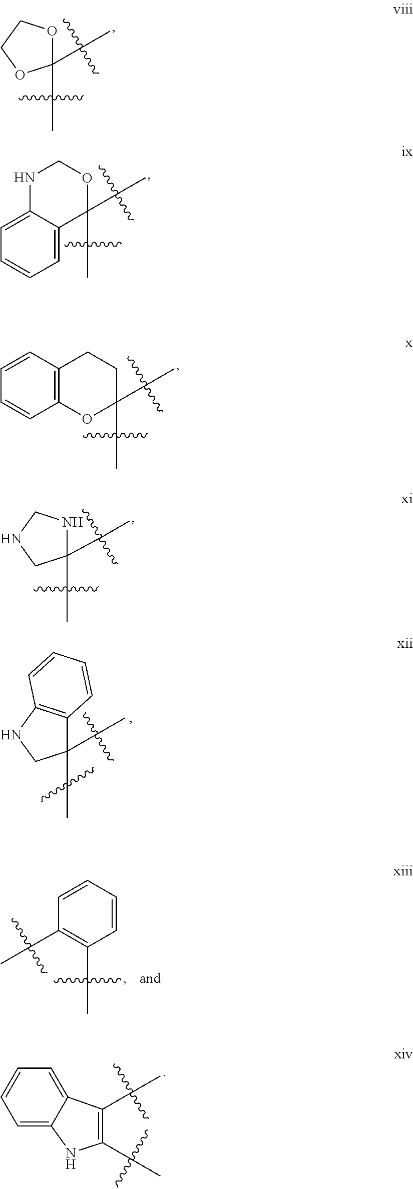
wherein each of viii-xiv optionally is substituted with 1-4 groups independently selected from R⁸ and -L-R⁸, and L and R⁸ are as defined in claim 1.

7. The compound of claim 1, or a pharmaceutically acceptable salt, hydrate, or ester thereof, wherein ring A optionally is substituted with 1-4 groups independently selected from oxo, ═NR⁶, ═CR⁶R⁷, —C(O)R⁵, —C(O)OR⁶, —C(O)NR⁶R⁷, —S(O)ₘR⁵, a C₁₋₁₀ alkyl group, a C₆₋₁₄ aryl group, a 3-14 membered cycloheteroalkyl group, and a 5-14 membered heteroaryl group, wherein each of the C₁₋₁₀ alkyl group, the C₆₋₁₄ aryl group, the 3-14 membered cycloheteroalkyl group, and the 5-14 membered heteroaryl group optionally is substituted with 1-4 groups independently selected from R⁸ and -L-R⁸, wherein m, L, R⁵, R⁶, R⁷, and R⁸ are as defined in claim 1.

8. The compound of claim 1, or a pharmaceutically acceptable salt, hydrate, or ester thereof, wherein ring A optionally is substituted with oxo, ═N—OH, or ═CR⁶R⁷; and R⁶ and R⁷, taken together with the carbon atom to which they are attached, form a C₃₋₁₄ cycloalkyl group, a C₆₋₁₄ aryl group, a 3-14 membered cycloheteroalkyl group, or a 5-14 membered heteroaryl group each optionally substituted with 1-4 groups independently selected from R⁸ and -L-R⁸, wherein L and R⁸ are as defined in claim 1.

9. The compound of claim 1, or a pharmaceutically acceptable salt, hydrate, or ester thereof, wherein ring A optionally is substituted with:

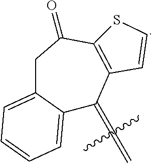

10. The compound of claim 1, or a pharmaceutically acceptable salt, hydrate, or ester thereof, wherein ring A optionally is substituted with 1-4 groups independently selected from —C(O)R⁵, —C(O)OR⁶, —C(O)NR⁶R⁷ and —SO₂R⁵, wherein R⁵, R⁶, and R⁷ are as defined in claim 1.

11. The compound of claim 10, or a pharmaceutically acceptable salt, hydrate, or ester thereof, wherein R⁵, R⁶, and R⁷, at each occurrence, independently are a C₁₋₁₀ alkyl group, a C₃₋₁₄ cycloalkyl group, or a C₆₋₁₄ aryl group, wherein each of the C₁₋₁₀ alkyl group, the C₃₋₁₄ cycloalkyl group, and the C₆₋₁₄ aryl group optionally is substituted with 1-4 -L-R⁸ groups, wherein L and R⁸ are as defined in claim 1.

12. The compound of claim 1, or a pharmaceutically acceptable salt, hydrate, or ester thereof, wherein ring A optionally is substituted with 1-4 groups independently selected from a C₁₋₁₀ alkyl group, a C₆₋₁₄ aryl group, a 3-14 membered cycloheteroalkyl group, and a 5-14 membered heteroaryl group, wherein each of the C₁₋₁₀ alkyl group, the C₆₋₁₄ aryl group, the 3-14 membered cycloheteroalkyl group, and the 5-14 membered heteroaryl group optionally is substituted with 1-4 groups independently selected from R⁸ and -L-R⁸, wherein L and R⁸ are as defined in claim 1.

13. The compound of claim 1, or a pharmaceutically acceptable salt, hydrate, or ester thereof, wherein ring A optionally is substituted with 1-4 groups independently selected from a methyl group, an ethyl group, a propyl group, and a benzyl group, each of which optionally is substituted with 1-4 -L-R⁸ groups, wherein L and R⁸ are as defined in claim 1.

14. The compound of claim 1, or a pharmaceutically acceptable salt, hydrate, or ester thereof, wherein ring A optionally is substituted with 1-4 groups independently selected from a C₆₋₁₄ aryl group, a 5-10 membered cycloheteroalkyl group, and a 5-10 membered heteroaryl group, each of which optionally is substituted with 1-4 groups independently selected from R⁸ and -L-R⁸, wherein L and R⁸ are as defined in claim 1.

15. The compound of claim 14, or a pharmaceutically acceptable salt, hydrate, or ester thereof, wherein the C₆₋₁₀ aryl group, the 5-10-membered cycloheteroalkyl group, and the 5-10 membered heteroaryl group are selected from a phenyl group, an imidazolidyl group, an isoxazolyl group, an oxadiazolyl group, a pyridyl group, an indolyl group, an indolinyl group, a benzofuranyl group, a tetrahydroisoquinolyl group, a 1H-benzoimidazolyl group, and a benzopyridyl group.

16. The compound of claim 1, or a pharmaceutically acceptable salt, hydrate, or ester thereof, wherein R² and R³ independently are selected from H, F, Cl, and Br.

17. The compound of claim 1, or a pharmaceutically acceptable salt, hydrate, or ester thereof, wherein R¹ is H.

18. The compound of claim 1, or a pharmaceutically acceptable salt, hydrate, or ester thereof, wherein the compound is in the form of an enantiomer.

19. A compound, or a pharmaceutically acceptable salt, hydrate, or ester thereof, the compound selected from:
4-[4-((E)-2-hydroxycarbamoyl-vinyl)-benzyl]-piperazine-1-carboxylic acid tent-butyl ester,
(E)-N-hydroxy-3-{4-[3-(1H-indol-3-yl)-piperidin-1-ylmethyl]-phenyl}-acrylamide,
(E)-N-hydroxy-3-{4-[(R)-3-(2-methyl-1H-indol-3-yl)-piperidin-1-ylmethyl]-phenyl}-acrylamide,
(E)-3-{3-fluoro-4-[3-(2-methyl-5-morpholin-4-yl-1H-indol-3-yl)-piperidin-1-ylmethyl]-phenyl}-N-hydroxy-acrylamide,
(E)-N-hydroxy-3-{4-[3-(2-phenyl-1H-indol-3-yl)-piperidin-1-ylmethyl]-phenyl}-acrylamide,
(E)-3-{4-[3-(2-tert-butyl-1H-indol-3-yl)-piperidin-1-ylmethyl]-phenyl}-N-hydroxy-acrylamide,
(E)-3-{4-[5-(2-tert-butyl-1H-indol-3-yl)-3,6-dihydro-2H-pyridin-1-ylmethyl]-phenyl}-N-hydroxy-acrylamide,
(E)-N-hydroxy-3-{4-[3-[(Z)-hydroxyimino]-5-(2-methyl-1H-indol-3-yl)-piperidin-1-ylmethyl]-phenyl}-acrylamide,
(E)-N-hydroxy-3-{4-[3-(2-oxo-2,3-dihydro-1H-indol-3-yl)-piperidin-1-ylmethyl]-phenyl}-acrylamide,
(E)-3-{3-fluoro-4-[3-(1H-indol-3-yl)-piperidin-1-ylmethyl]-phenyl}-N-hydroxy-acrylamide,
(E)-3-{4-[(S)-3-(2-tert-butyl-1H-indol-3-yl)-piperidin-1-ylmethyl]-3-fluoro-phenyl}-N-hydroxy-acrylamide,
(E)-3-{4-[3-(2-tert-butyl-1H-indol-3-yl)-piperidin-1-ylmethyl]-3-chloro-phenyl}-N-hydroxy-acrylamide,
(E)-3-(3-fluoro-4-{3-[2-(1-hydroxy-1-methyl-ethyl)-1H-indol-3-yl]-piperidin-1-ylmethyl}-phenyl)-N-hydroxy-acrylamide,
(E)-3-{3-fluoro-4-[3-(2-isopropenyl-1H-indol-3-yl)-piperidin-1-ylmethyl]-phenyl}-N-hydroxy-acrylamide,
(E)-3-{3-fluoro-4-[3-(2-phenyl-1H-indol-3-yl)-piperidin-1-ylmethyl]-phenyl}-N-hydroxy-acrylamide,
N-hydroxy-3-{4-[3-(2-phenyl-1H-indol-3-yl)-piperidin-1-ylmethyl]-phenyl}-propionamide,
3-{1-[2-fluoro-4-((E)-2-hydroxycarbamoyl-vinyl)-benzyl]-piperidin-3-yl}-1H-indole-2-carboxylic acid ethyl ester,
3-{1-[2-fluoro-4-((E)-2-hydroxycarbamoyl-vinyl)-benzyl]-piperidin-3-yl}-1H-indole-2-carboxylic acid hydroxyamide,
(E)-3-(3-fluoro-4-{3-[2-(morpholine-4-carbonyl)-1H-indol-3-yl]-piperidin-1-ylmethyl}-phenyl)-N-hydroxy-acrylamide,
(E)-N-hydroxy-3-{4-[3-(2-methyl-5-morpholin-4-yl-1H-indol-3-yl)-piperidin-1-ylmethyl]-phenyl}-acrylamide,
3-{3-fluoro-4-[3-(2-isopropyl-1H-indol-3-yl)-piperidin-1-ylmethyl]-phenyl}-N-hydroxy-propionamide,
(E)-3-[4-(1,1-dimethyl-1,3,4,9-tetrahydro-beta-carbolin-2-ylmethyl)-phenyl]-N-hydroxy-acrylamide,
(E)-N-hydroxy-3-[4-(1-oxo-1,3,4,9-tetrahydro-beta-carbolin-2-ylmethyl)-phenyl]-acrylamide,
(E)-N-hydroxy-3-{4-[2-(3-phenyl-isoxazol-5-yl)-morpholin-4-ylmethyl]-phenyl}-acrylamide,
(E)-N-hydroxy-3-{4-[2-(3-phenyl-[1,2,4]oxadiazol-5-yl)-morpholin-4-ylmethyl]-phenyl}-acrylamide,
(E)-3-[4-(4-benzyl-piperazin-1-ylmethyl)-phenyl]-N-hydroxy-acrylamide,
(E)-3-{4-[4-(4-bromo-benzyl)-piperazin-1-ylmethyl]-phenyl}-N-hydroxy-acrylamide,
4-[4-((E)-2-hydroxycarbamoyl-vinyl)-benzyl]-piperazine-1-carboxylic acid ethyl ester,
4-[4-((E)-2-hydroxycarbamoyl-vinyl)-benzyl]-piperazine-1-carboxylic acid benzyl ester,
(E)-N-hydroxy-3-{4-[4-(1-methyl-6-oxo-1,6-dihydro-pyridin-2-yl)-piperazin-1-ylmethyl]-phenyl}-acrylamide,
(E)-3-{4-[4-(1-benzoyl-1H-benzoimidazol-2-yl)-piperazin-1-ylmethyl]-phenyl}-N-hydroxy-acrylamide,
(E)-3-{4-[4-(3-chloro-isoquinolin-1-yl)-piperazin-1-ylmethyl]-phenyl}-N-hydroxy-acrylamide,
(E)-N-hydroxy-3-(4-piperidin-1-ylmethyl-phenyl)-acrylamide,
(E)-3-spiro[4H-3,1-benzoxazine-4,4′-piperidin]-2(1H)-1-ylmethyl)-phenyl]-N-hydroxy-acrylamide,
(E)-3-[4-(4-benzofuran-2-yl-piperidin-1-ylmethyl)-phenyl]-N-hydroxy-acrylamide,
(E)-3-{4-[4-(7-bromo-5-methoxy-benzofuran-2-yl)-piperidin-1-ylmethyl]-phenyl}-N-hydroxy-acrylamide,
(E)-3-[4-(3-cyclopropylmethyl-2,4-dioxo-1,3,8-triaza-spiro[4.5]dec-8-ylmethyl)-phenyl]-N-hydroxy-acrylamide,
(E)-N-hydroxy-3-(4-(spiro[chroman-2,4′-piperidine]-1′-ylmethyl)phenyl)acrylamide,
(E)-N-hydroxy-3-(4-((1-methylsulfonyl)spiro[indoline-3,4′-piperidine]-1′-yl)methyl)phenyl)acrylamide,
(E)-3-(4-{4-[4-chloro-2-(2-oxo-pyrrolidin-1-ylmethyl)-phenyl]-piperidin-1-ylmethyl}-phenyl)-N-hydroxy-acrylamide,
(E)-N-hydroxy-3-{4-[4-(1-oxo-3,4-dihydro-1H-isoquinolin-2-yl)-piperidin-1-ylmethyl]-phenyl}-acrylamide,
(E)-3-(4-{4-[(acetyl-methyl-amino)-methyl]-4-phenyl-piperidin-1-ylmethyl}-phenyl)-N-hydroxy-acrylamide,
(E)-N-hydroxy-3-(4-((1-methyl-2-oxospiro[indoline-3,4′-piperidine]-1′-yl)methyl)phenyl)acrylamide,
(E)-N-hydroxy-3-{4-[4-(2-oxo-imidazolidin-1-yl)-piperidin-1-ylmethyl]-phenyl}-acrylamide,
(E)-3-{4-[4-(4-chloro-phenyl)-4-hydroxycarbamoylmethyl-piperidin-1-ylmethyl]-phenyl}-N-hydroxy-acrylamide,
(E)-N-hydroxy-3-{4-[4-(10-oxo-9,10-dihydro-1-thia-benzo[f]azulen-4-ylidene)-piperidin-1-ylmethyl]-phenyl}-acrylamide,
(E)-N-hydroxy-3-(4-morpholin-4-ylmethyl-phenyl)-acrylamide,
(E)-3-[4-(1,4-dioxa-8-aza-spiro[4.5]dec-8-ylmethyl)-phenyl]-N-hydroxy-acrylamide,
(E)-3-[4-(4-benzyl-piperidin-1-ylmethyl)-phenyl]-N-hydroxy-acrylamide,
(E)-3-[4-(4-benzenesulfonyl-piperazin-1-ylmethyl)-phenyl]-N-hydroxy-acrylamide,
(E)-3-[4-(4-benzoyl-piperazin-1-ylmethyl)-phenyl]-N-hydroxy-acrylamide,
(E)-N-hydroxy-3-[4-(4-propionyl-piperazin-1-ylmethyl)-phenyl]-acrylamide,
(E)-3-{4-[4-(3,3-dimethyl-butyryl)-piperazin-1-ylmethyl]-phenyl}-N-hydroxy-acrylamide,
(E)-3-[4-(4-cyclohexanecarbonyl-piperazin-1-ylmethyl)-phenyl]-N-hydroxy-acrylamide,
(E)-N-hydroxy-3-{4-[4-(toluene-4-sulfonyl)-piperazin-1-ylmethyl]-phenyl}-acrylamide,
(E)-N-hydroxy-3-{4-[4-(toluene-3-sulfonyl)-piperazin-1-ylmethyl]-phenyl}-acrylamide,
(E)-3-{4-[4-(4-fluoro-benzenesulfonyl)-piperazin-1-ylmethyl]-phenyl}-N-hydroxy-acrylamide,
(E)-3-[4-(4-benzenesulfonyl-3,4-dihydro-2H-quinoxalin-1-ylmethyl)-phenyl]-N-hydroxy-acrylamide,
4-[4-((E)-2-hydroxycarbamoyl-vinyl)-benzyl]-3,4-dihydro-2H-quinoxaline-1-carboxylic acid phenylamide,
(E)-3-[4-(4-cyclohexanecarbonyl-3,4-dihydro-2H-quinoxalin-1-ylmethyl)-phenyl]-N-hydroxy-acrylamide, and
4-[4-((E)-2-hydroxycarbamoyl-vinyl)-benzyl]-3,4-dihydro-2H-quinoxaline-1-carboxylic acid benzylamide.

20. A composition comprising a therapeutically effective amount of a compound of claim 1, or a pharmaceutically acceptable salt, hydrate, or ester thereof, and a pharmaceutically acceptable carrier or excipient.

21. A method of inhibiting a deacetylase in a cell, the method comprising contacting a cell with a compound of claim 1, or a pharmaceutically acceptable salt, hydrate, or ester thereof, in an amount sufficient to inhibit a deacetylase.

22. (canceled)

23. A method of treating a disease, disorder, condition, or undesired process mediated by a deacetylase in a mammal, the method comprising administering to a mammal a therapeutically effective amount of a compound of claim 1, or a pharmaceutically acceptable salt, hydrate, or ester thereof.

24. (canceled)

25. (canceled)

26. The method of claim 23, wherein the deacetylase is a histone deacetylase.

27. The method of claim 26, wherein the disease, disorder, condition, or undesired process is selected from an undesired proliferative condition, a neurodegenerative disease, a cardiovascular disease, stroke, an autoimmune disease, an inflammatory disorder, an undesired immunological process, and a fungal infection.

28. The method of claim 27, wherein the disease, disorder, condition, or undesired process is selected from a cancer, a tumor, a fibrosis, a neoplasia, psoriasis, prostate hyperplasia, Alzheimer's disease, Huntington's disease, Rubenstein-Taybis syndrome, Parkinson's disease, muscular dystrophy, heart failure, cardiac hypertrophy, thrombosis, spinal muscular atrophy, stroke, Rett's syndrome, Lupus, scleroderma, atherosclerosis, and an arthritis or arthritic condition.

29. The method of claim 28, wherein the cancer is selected from brain cancer, kidney cancer, liver cancer, adrenal gland cancer, bladder cancer, breast tumor, stomach cancer, esophagus cancer, ovarian cancer, colon cancer, rectum cancer, prostate cancer, pancrea cancer, lung cancer, vagina cancer, thyroid cancer, sarcoma, glioblastomas, multiple myeloma, gastrointestinal cancer, breast cancer, and leukemia.

30. The method of claim 29, wherein the mammal is a human.
